(19) United States
(12) Patent Application Publication
(10) Pub. No.: US 2003/0078279 A1
(43) Pub. Date: Apr. 24, 2003

(54) Spiropiperidine compounds as ligands for ORL-1receptor

(75) Inventors: Fumitaka Ito, Taketoyo (JP); Hiroki Koike, Ohbu (JP); Masaki Sudo, Handa (JP); Tatsuya Yamagishi, Handa (JP); Koji Ando, Handa-shi (JP)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/153,310

(22) Filed: May 22, 2002

Related U.S. Application Data
(60) Provisional application No. 60/301,079, filed on Jun. 26, 2001.

(51) Int. Cl. A61K 31/4747; C07D 471/12

(52) U.S. Cl. 514/278; 546/17

(57) ABSTRACT

A compound of the formula:

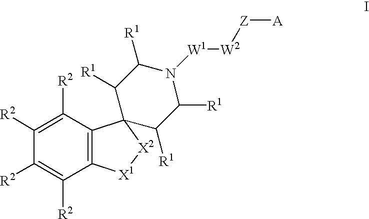

or a salt, prodrug or solvate thereof, wherein R¹ and R² groups are all hydrogen; A is a benzofuzed azahetero ring; W¹-W² is CH₂-CH₂; X¹-X² is CH₂-CH₂; and Z is methylene or carbonyl; or the like, is a ligand for ORL1-receptor and are useful for treating or preventing pain, a CNS disorder or the like in mammalian subjects.

### TECHNICAL FIELD

This invention relates to substituted spiropiperidine compounds and their salts, prodrugs and solvates, and a medical use thereof. Also, this invention relates to a pharmaceutical composition comprising said compound, or its salt, prodrug or solvate. The compounds of this invention have binding affinity for ORL-1 receptor. In particular, compounds of this invention have selective antagonist activity for said receptor. The compounds of this invention are useful in treating or preventing disorders or medical conditions selected from pain, a CNS disorder and the like, which is mediated by said receptor and its endogeneous ligand.

### BACKGROUND ART

Three types of opioid receptors, μ (mu), δ (delta) and κ (kappa) have been identified. These receptors may be indicated with combinations of OP (abbreviation for Opioid Peptides) and numeric subscripts as suggested by the International Union of Pharmacology (IUPHAR). Namely, OP₁, OP₂ and OP₃ respectively correspond to δ-, κ- and μ-receptors. It has been found out that they belong to G-protein-coupled receptors and distribute in the central nervous system (CNS), peripheries and organs in a mammal. As ligands for the receptors, endogeneous and synthetic opioids are known. It is believed that an endogeneous opioid peptide produces their effects through an interaction with the major classes of opioid receptors. For example, endorphins have been purified as endogeneous opioid peptides and bind to both δ- and μ-receptors. Morphine is a well-known non-peptide opioid analgesic and has binding affinity mainly for μ-receptor. Opiates have been widely used as pharmacological agents, but drugs such as morphine and heroin induce some side effects such as drug addiction and euphoria.

Further, Meunier et al. reported isolation of a seventeen-amino-acid-long peptide from rat brain as an endogeneous ligand for an orphan opioid receptor (Nature, Vol. 337, pp. 532-535, Oct. 12, 1995). The receptor is known as “opioid receptor-like 1 (abbreviated as ORL1-receptor)” which is believed to be almost as homologous to any of μ-, δ- and κ-receptors. In the same report, the endogeneous opioid ligand has been introduced as agonist for ORL-1 receptor and named as “nociceptine (abbreviated as NC)”. Also, the same ligand was named as “orphanin FQ (abbreviated as OFQ or oFQ)” by Reinscheid et al. (Science, Vol. 270, pp. 792-794, 1995). This receptor may be indicated as OP₄ in line with a recommendation by IUPHAR in 1998 (British Journal of Pharmacology, Vol. 129, pp. 1261-1283, 2000).

Opioids and their affinity for these receptors have been researched in-vitro and in-vivo. It is possible to date to test whether an opioid has agonist or antagonist properties or a combination of both on the receptors.

Use of a synthetic ORL1 -receptor ligand or antagonist as an analgesic is disclosed in WO 00/27815 (Smithkline Beecham Spa) or WO 99/48492 (Japan Tobacco Inc.).

Use of a synthetic ORL1-receptor antagonist for treating a CNS disorder is disclosed in WO 00/27815 (Smithkline Beecham Spa), WO 99/29696 (F. Hoffmann-La Roche AG) or British Journal of Pharmacology, Vol. 129, pp. 1261-1283, 2000 by G. Calo et al.

Banyu's WO 98/54168, WO 00/31061, WO 00/34280 and Japanese Patent Publication Kokai 2000-169476 disclose use of a synthetic ORL1-receptor ligand or antagonist as an analgesic or for treating a CNS disorder.

Schering's WO 01/07051 discloses use of a synthetic ORL-1 agonist in treating cough.

### BRIEF DISCLOSURE OF THE INVENTION

The present invention provides a compound of the following formula:

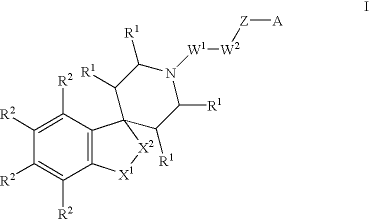

or salts thereof, wherein

each R¹ is independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}R^{a2}, R^{a3} and R^{a4} are independently selected from hydrogen, (C₁-C₆)alkyl, [(C₁-C₆)alkyl]—C(═O)—, [(C₁-C₆)alkoxy]-C(═O)— and [(C₁-C₆)alkyl]-SO₂—; or

two R¹ groups taken together form —CH₂— or —(CH₂)₂— and the remaining R¹ groups are defined as above;

each R² is independently selected from

hydrogen;

halo;

hydroxy;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;

(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;

non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—;

aryl selected from phenyl and naphthyl; and

four- to eight-membered heterocyclyl containing one to four hetero atoms in the ring independently selected from nitrogen, oxygen and sulfur;

X¹ and X² are independently selected from

(CH₂)ₙ₁ wherein n1 is an integer selected from 1, 2 and 3;

O;

NH;

S;

C(═O);

SO₂;

NR^{X1};

N—C(═O)R^{X2};

N—C(═O)OR^{X3}; and

N—C(═O)NR^{X4}R^{X5};

wherein

R^{X1}, R^{X2}, R^{X3}, R^{X4} and R^{X5} are independently (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}— and R^{a3}R^{a3}R^{a4}N-C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or

X¹ and X² taken together form CH═CH;

W¹ and W² are independently selected from CR^{W1}R^{W2},

wherein

R^{W1} and R^{W2} are independently selected from

hydrogen;

halo;

hydroxy;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;

(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;

C(═O)—[(C₁-C₆)alkyl] wherein said (C₁-C₆)alkyl is optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;

C(═O)—NR^{W11}R^{W12} wherein R^{W11} and R^{W12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;

NR^{W13}R^{W14} wherein R^{W13} and R^{W14} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;

aryl selected from phenyl and naphthyl; and

four- to eight-membered heterocyclyl containing one to four hetero atoms independently selected from nitrogen, oxygen and sulfur;

A is selected from AA; AB; AC and AD:

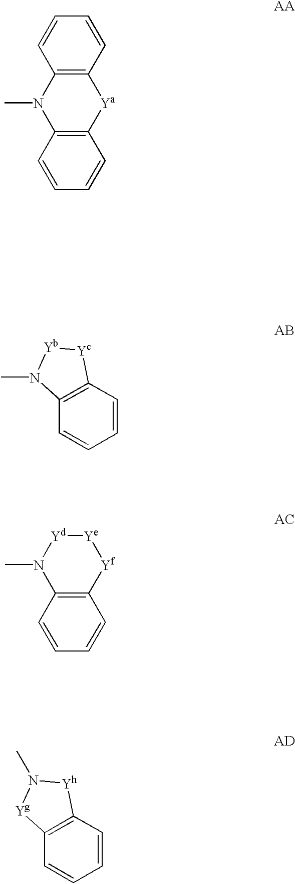

wherein

Y^{a} is selected from (CH₂)ₙ₂ wherein n2 is an integer selected from 0, 1 and 2; C(═O); NH; O and S;

Y^{b}, Y^{c}, Y^{d}, Y^{e}, Y^{f}, Y^{g} and Y^{h} are independently selected from

C(═O);

CR^{Y1}R^{Y2};

CR^{Y3}[C(═O)R^{Y4}];

CR^{Y3}NR^{Y5}C(═O)R^{Y4}];

CR^{Y3}[C(═O)NR^{Y6}R^{Y7}];

CR^{Y3}[NR^{Y6}R^{Y7}];

O;

S;

SO₂;

NH;

N[(C₁-C₆)alkyl] wherein said (C₁-C₆)alkyl is optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;

N—(CH₂)ₙ₃-heterocyclyl wherein n3 is an integer selected from 0, 1, 2 and 3, and said heterocyclyl contains from four to eight ring atoms one or two of which are independently selected from nitrogen, oxygen and sulfur;

N—(CH₂)ₙ₄-aryl wherein n4 is an integer selected from 0, 1, 2 and 3, and said aryl is selected from phenyl and naphthyl; and

N—(CH₂)ₙ₅-heteroaryl wherein n5 is an integer selected from 0, 1, 2 and 3, and said heteroaryl is a five to ten membered aromatic heterocyclyl containing from one to four hetero atoms independently selected from nitrogen, oxygen and sulfur; or

Y^{b} and Y^{c} taken together form a group selected from CR^{Y81}═CR^{Y82}; CR^{Y83}═N and N═N; and Y^{d}, Y^{e}, Y^{f}, Y^{g} and Y^{h} are defined as above; wherein

R^{Y1}, R^{Y2} and R^{Y5} are independently selected from

hydrogen;

hydroxy;

non-, mono- and di-substituted amino wherein the substituents are independently selected from (C₁-C₆)alkyl; [(C₁-C₆)alkyl]-C(═O)—; [(C₁-C₆)alkoxy]-C(═O)—; [(C₁-C₆)alkyl]-SO₂—; and four- to eight-membered heterocyclyl containing one to four hetero atoms independently selected from nitrogen, oxygen and sulfur, wherein said heterocyclyl is optionally substituted with one to three substituents independently selected from hydroxy, (C₁-C₆)alkyl, NH₂—C(O═)—, [(C₁-C₆)alkyl]-NH—C(═O)—, [(C₁-C₆)alkyl]₂-N—C(═O)—, and non-, mono- and di-substituted amino wherein the substituents are independently selected from (C₁-C₆)alkyl, [(C₁-C₆)alkyl]C(═O)—, [(C₁-C₆)alkoxy]-C(═O)— and [(C₁-C₆)alkyl]-SO₂—;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxyl-C(═O)— and [(C₁-C₆)alkyl]-SO₂—; or

R^{Y1} and R^{Y2} taken together with the carbon atom to which they are attached form spiropyrrolidinyl or spiropiperidinyl, both of which are optionally N-substituted with a substituent selected from (C₁-C₆)alkyl, (C₁-C₆)alkyl-C(═O)—, [(C₁-C₆)alkyl]-C(═O)—(C₁-C₆)alkyl and aryl-(C═O)— wherein aryl is selected from phenyl and naphthyl; and R^{Y5} is defined as above;

R^{Y3} is hydrogen;

R^{Y4} is selected from

hydroxy;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

R^{Y6} and R^{Y7} are independently selected from

hydrogen;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and C₆)alkyl]-SO₂—;

hetrocyclyl-(CH₂)ₙ₆— wherein n6 is an integer selected from 0, 1, 2, 3 and 4 and said heterocyclyl is four to eight membered containing one to three hetero atoms independently selected from nitrogen, oxygen and sulfur, wherein said heterocyclyl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C (═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and

hetroaryl-(CH₂)ₙ₇— wherein n7 is an integer selected from 0, 1, 2, 3 and 4 and said heteroaryl is five to ten membered containing one to three hetero atoms independently selected from nitrogen, oxygen and sulfur, wherein said heteroaryl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; or

R^{Y6} and R^{Y7} taken together with the nitrogen atom to which they are attached form a four to eight heterocyclyl optionally containing, in addition to the nitrogen atom, one to two additional hetero atoms independently selected from nitrogen, oxygen and sulfur, and said heterocyclyl is optionally substituted with one substituent selected from hydroxy; (C₁-C₆)alkyl; NH₂-C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxyl-C(═O)— and [(C₁-C₆)alkyl]-SO₂—;

R^{Y81}, R^{Y82} and R^{Y83} are independently selected from R^{Y811} and R^{Y812}C(═O)— wherein R^{Y811} and R^{Y812} are independently selected from

hydrogen;

hydroxy;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

said A is optionally substituted in the fused benzene rings with one to four substituents independently selected from

halo;

hydroxy;

mercapto;

phenyl;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxyl-C(═O)— and [(C₁-C₆)alkyl]-SO₂—; and

(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

Z is selected from

C(═O);

(CH₂)ₙ₈ wherein n8 is an integer selected from 0, 1 and 2; and

CHR^{Z1} wherein

R^{Z1} is selected from carboxy;

(C₁-C₆)alkoxy-C(═O)—;

non-, mono- and di-substituted amino wherein the substituents are independently selected from (C₁-C₆)alkyl, [(C₁-C₆)alkyl]-C(═O)—, [(C₁-C₆)alkyl]-C(═O)—O— and [(C₁-C₆)alkyl]-SO₂—;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

[C(═O)—NR^{Z11}R^{Z12}] wherein R^{Z11} and R^{Z12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C6)alkyl]-C(═O)—, [(C₁-C₆)alkoxy]-C(═O)— and [(C₁-C₆)alkyl]-SO₂—.

The compounds of the present invention have binding affinity for opioid receptor-like 1 (hereinafter referred to as “ORL-1 receptor”).

It is therefore an object of the present invention to provide a compound of formula I which is useful as a lignad for ORL-1 receptor.

It is another object of the present invention to provide a compound of formula I which is a modulator of ORL-1 receptor.

It is another object of the present invention to provide a compound of formula I having selective affinity for ORL-1 receptor. Preferably, these compounds have selective affinity for ORL-1 receptor than μ-receptor.

It is another object of the present invention to provide a compound of formula I having antagonist activity for ORL-1 receptor.

It is another object of the present invention to provide a compound of formula I having selectivity for ORL-1 receptor and antagonist effect for said receptor.

The present invention relates to use of a compound of formula I as a ligand or a modulator for ORL-1 receptor, preferably as a selective ligand for said receptor, more preferably as an antagonist for said receptor, and most preferably as a selective antagonist for said receptor.

### DETAILED DESCRIPTION OF THE INVENTION

The term “pain” as used herein includes acute and chronic pain; neuropathic or inflammatory pain such as post herpetic neuralgia, neuralgia, diabetic neuropathy or post operative pain; osteoarthritis or back pain; pain in pregnancy labor and pains known to those skilled in the art (e.g., the pains described in Advances in Pain Research and Therapy, edited by C. R. Chapman et al., and published by Ravan Press (1989)).

The term “alkyl”, as used herein, means a straight or branched saturated monovalent hydrocarbon radical including, but not limited to, methyl, ethyl, propyl, isopropyl, n-butyl, sec-butyl, tert-butyl and the like.

The term “cycloalkyl”, as used herein, means a saturated carbocyclic radical including, but not limited to, cyclopropyl, cyclobutyl, cyclopentyl, cyclohexyl, cycloheptyl, cyclooctyl, cyclononyl, cyclodecyl and the like.

The term “alkoxy”, as used herein, means an O-alkyl group wherein “alkyl” is defined above.

The term “halo”, as used herein, refers to F, Cl, Br or I, preferably F or Cl.

The term “treating”, as used herein, refers to reversing, alleviating, inhibiting the progress of, or preventing the disorder or condition to which such term applies, or one or more symptoms of such disorder or condition. The term “treatment” as used herein refers to the act of treating, as “treating” is defined immediately above.

A preferred class of compound of formula (I) of this invention is that wherein:

all R¹ are hydrogen

each R² is independently selected from hydrogen and halo;

X¹ is selected from (CH₂)ₙ₁ wherein n1 is an integer selected from 1, 2 and 3; O; NH; S; C(═O); SO₂; and N[(C₁-C₄)alkyl];

X² is selected from CH₂; O; NH; S; C(═O); SO₂; and N[(C₁-C₄)alkyl]; or

X¹ and X² taken together form CH═CH;

W¹ and W² are independently selected from CR^{W1}R^{W2},

wherein

R^{W1} and R^{W2} are independently selected from

hydrogen;

halo;

hydroxy;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxyl-C(═O)— and [(C₁-C₆)alkyl]-SO₂—;

(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;

C(═O)—[(C₁-C₆)alkyl] wherein said (C₁-C₆)alkyl is optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;

C(═O)—NR^{W11}R^{W12} wherein R^{W11} and R^{W12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a1}R^{a2}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;

NR^{W13}R^{W14} wherein R^{W13} and R^{W14} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;

aryl selected from phenyl and naphthyl; and

four- to eight-membered heterocyclyl containing one to four hetero atoms independently selected from nitrogen, oxygen and sulfur;

A is AB wherein

Y^{b} and Y^{c} are independently selected from

C(═O);

CR^{Y1}R^{Y2};

CR^{Y3}[C(═O)R^{Y4}];

CR^{Y3}[C(═O)NR^{Y6}R^{Y7}];

CR^{Y3}[NR^{Y6}R^{Y7})];

O;

S;

SO₂;

NH;

N[(C₁-C₆)alkyl] wherein said (C₁-C₆)alkyl is optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;

N—(CH₂)ₙ₃-heterocyclyl wherein n3 is an integer selected from 0, 1, 2 and 3, and said heterocyclyl contains from four to eight ring atoms one or two of which are independently selected from nitrogen, oxygen and sulfur;

N—(CH₂)ₙ₄-aryl wherein n4 is an integer selected from 0, 1, 2 and 3, and said aryl is selected from phenyl and naphthyl; and

N—(CH₂)ₙ₅-heteroaryl wherein n5 is an integer selected from 0, 1, 2 and 3, and said heteroaryl is a five to ten membered aromatic heterocyclyl containing from one to four hetero atoms independently selected from nitrogen, oxygen and sulfur; or

Y^{b} and Y^{c} taken together form a group selected from CR^{Y81}═CR^{Y82}; CR^{Y83}═N and N═N; and Y^{d}, Y^{e}, Y^{f}, Y^{g} and Y^{h} are defined as above;

R^{Y1} and R^{Y2} are independently selected from

hydrogen;

hydroxy;

non-, mono- and di-substituted amino wherein the substituents are independently selected from (C₁-C₆)alkyl; [(C₁-C₆)alkyl]-C(═O)—; [(C₁-C₆)alkoxy]-C(═O)—; [(C₁-C₆)alkyl]-SO₂—; and four- to eight-membered heterocyclyl containing one to four hetero atoms independently selected from nitrogen, oxygen and sulfur, wherein said heterocyclyl is optionally substituted with one to three substituents independently selected from hydroxy, (C₁-C₆)alkyl, NH₂—C(O═)—, [(C₁-C₆)alkyl]-NH—C(═O)—, [(C₁-C₆)alkyl]₂-N—C(═O)—, and non-, mono- and di-substituted amino wherein the substituents are independently selected from (C₁-C₆)alkyl, [(C₁-C₆)alkyl]-C(═O)—, [(C-C₆)alkoxy]-C(═O)— and [(C₁-C₆)alkyl]-SO₂—;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁C₆)alkyl]-SO₂—; or

R^{Y1} and R^{Y2} taken together with the carbon atom to which they are attached form spiropyrrolidinyl or spiropiperidinyl, both of which are optionally N-substituted with a substituent selected from (C₁-C₆)alkyl, (C₁-C₆)alkyl-C(═O)—, [(C₁-C₆)alkyl]-C(═O)—(C₁-C₆)alkyl and aryl-(C═O)— wherein aryl is selected from phenyl and naphthyl;

R^{Y3} is hydrogen;

R^{Y4} is selected from

hydroxy;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

R^{Y5}, R^{Y6} and R^{Y7} are independently selected from

hydrogen;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [C₁-C₆)alkyl]-SO₂—;

hetrocyclyl-(CH₂)ₙ₆— wherein n6 is an integer selected from 0, 1, 2, 3 and 4 and said heterocyclyl is four to eight membered containing one to three hetero atoms independently selected from nitrogen, oxygen and sulfur, wherein said heterocyclyl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and

hetroaryl-(CH₂)ₙ₇— wherein n7 is an integer selected from 0, 1, 2, 3 and 4 and said heteroaryl is five to ten membered containing one to three hetero atoms independently selected from nitrogen, oxygen and sulfur, wherein said heteroaryl is optionally substituted with one to three substituents independently selected from hydroxy; (C₁-C₆)alkyl; NH₂-C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; or

R^{Y6} and R^{Y7} taken together with the nitrogen atom to which they are attached form a four to eight heterocyclyl optionally containing, in addition to the nitrogen atom, one to two additional hetero atoms independently selected from nitrogen, oxygen and sulfur, and said heterocyclyl is optionally substituted with one substituent selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—;

R^{Y81}, R^{Y82} and R^{Y83} are independently selected from R^{Y811} and R^{Y812}C(═O)— wherein R^{Y811} and R^{Y812} are independently selected from

hydrogen;

hydroxy;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a6}R^{a7}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)—and [(C₁-C₆)alkyl]-SO₂—; and

said A is optionally substituted in the fused benzene rings with one to four substituents independently selected from

halo;

hydroxy;

mercapto;

phenyl;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

(C₁-C₆)alkoxy optionally substituted with one to three substituents independently selected from halo, hydroxy, carboxy, [(C₁-C₆)alkyl]-C(═O)-, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

Z is selected from

C(═O);

(CH₂)ₙ₈ wherein n8 is an integer selected from 0, 1 and 2; and

CHR^{Z1} wherein

R^{Z1} is selected from

carboxy;

(C₁-C₆)alkoxy-C(═O)—;

non-, mono- and di-substituted amino wherein the substituents are independently selected from (C₁-C₆)alkyl, [(C₁-C₆)alkyl]-C(═O)—, [(C₁-C₆)alkyl]-C(═O)—O— and [(C₁-C₆)alkyl]-SO₂—;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

[C(═O)—NR^{Z11}R^{Z12}] wherein R^{Z11} and R^{Z12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—.

A further preferred class of compound of formula (I) of this invention is that wherein:

all R¹ are hydrogen

each R² is independently selected from hydrogen and halo;

X¹ is selected from (CH₂)ₙ₁ wherein n1 is an integer selected from 1, 2 and 3; O; NH; S; C(═O); SO₂; and N[(C₁-C₄)alkyl];

X² is selected from CH₂; O; NH; S; C(═O); SO₂; and N[(C₁-C₄)alkyl]; or

X¹ and X² taken together form CH═CH;

W¹ and W² are both CH₂;

A is AB wherein

both Y^{b} and Y^{c} are independently selected from

C(═O);

CR^{Y1}R^{Y2};

CR^{Y3}[C(═O)R^{Y4}];

CR^{Y3}[C(═O)NR^{Y6}R^{Y7}]; and

CR^{Y3}[NR^{Y6}R^{Y7}], wherein

R^{Y1} and R^{Y2} are independently selected from

hydrogen;

hydroxy;

non-, mono- and di-substituted amino wherein the substituents are independently selected from (C₁-C₆)alkyl; [(C₁-C₆)alkyl]-C(═O)—; [(C₁-C₆)alkoxy]-C(═O)—; [(C₁-C₆)alkyl]-SO₂—; and four- to eight-membered heterocyclyl containing one to four hetero atoms independently selected from nitrogen, oxygen and sulfur, wherein said heterocyclyl is optionally substituted with one to three substituents independently selected from hydroxy, (C₁-C₆)alkyl, NH₂-C(O═)—, [(C₁-C₆)alkyl]-NH—C(═O)—, [(C₁-C₆)alkyl]₂-N—C(═O)—, and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or

R^{Y1} and R^{Y2} taken together with the carbon atom to which they are attached form spiropyrrolidinyl or spiropiperidinyl, both of which are optionally N-substituted with a substituent selected from (C₁-C₆)alkyl, (C₁-C₆)alkyl-C(═O)—, [(C₁-C₆)alkyl]-C(═O)—(C₁-C₆)alkyl and aryl-(C═O)— wherein aryl is selected from phenyl and naphthyl;

R^{Y3} is hydrogen;

R^{Y4} is selected from hydroxy;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxyl-C(═O)— and [(C₁-C₆)alkyl]-SO₂—; and

R^{Y6} and R^{Y7} are independently selected from hydrogen;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;

hetrocyclyl-(CH₂)ₙ₆— wherein n6 is an integer selected from 0, 1, 2, 3 and 4 and said heterocyclyl is four to eight membered containing one to three hetero atoms independently selected from nitrogen, oxygen and sulfur, wherein said heterocyclyl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and

hetroaryl-(CH₂)ₙ₇— wherein n7 is an integer selected from 0, 1, 2, 3 and 4 and said heteroaryl is five to ten membered containing one to three hetero atoms independently selected from nitrogen, oxygen and sulfur, wherein said heteroaryl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; or

R^{Y6} and R^{Y7} taken together with the nitrogen atom to which they are attached form a four to eight heterocyclyl optionally containing, in addition to the nitrogen atom, one to two additional hetero atoms independently selected from nitrogen, oxygen and sulfur, and said heterocyclyl is optionally substituted with one substituent selected from hydroxy; (C₁-C₆)alkyl; NH₂-C(O=)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—;

said A is optionally substituted in the fused benzene rings with one to four substituents independently selected from

halo;

hydroxy;

mercapto;

phenyl;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [C₁-C₆)alkyl]-SO₂—; and

(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

Z is selected from

C(═O);

(CH₂)ₙ₈ wherein n8 is an integer selected from 0, 1 and 2; and

CHR^{Z1} wherein

R^{Z1} is selected from

carboxy;

(C₁-C₆)alkoxy-C(═O)—;

non-, mono- and di-substituted amino wherein the substituents are independently selected from (C₁-C₆)alkyl, [(C₁-C₆)alkyl]-C(═O)—, [(C₁-C₆)alkyl]-C(═O)—O— and [(C₁-C₆)alkyl]-SO₂—;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxyl-C(═O)— and [(C₁-C₆)alkyl]-SO₂—; and

[C(═O)—NR^{Z11}R^{Z12}] wherein R^{Z11} and R^{Z12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—.

A further preferred class of compound of formula (I) of this invention is that wherein

all R¹ are hydrogen

each R² is independently selected from hydrogen and halo;

X¹ is selected from (CH₂)ₙ₁ wherein n1 is an integer selected from 1, 2 and 3; O; NH; S; C(═O); SO₂; and N[(C₁-C₄)alkyl];

X is selected from CH₂; O; NH; S; C(═O); SO₂; and N[(C₁-C₄)alkyl]; or

X¹ and X² taken together form CH═CH;

W¹ and W² are both CH₂;

A is AB wherein

Y^{b} is CR^{Y3}[C(═O)NR^{Y6}R^{Y7}]; and

Y^{c} is selected from

CR^{Y1} R^{Y2};

CR^{Y3}[C(═O)R^{Y4}];

CR^{Y3}[C(═O)NR^{Y6}R^{Y7}]; and

CR^{Y3}[NR^{Y6}R^{Y7}], wherein

R^{Y1} and R^{Y2} are independently selected from

hydrogen;

hydroxy;

non-, mono- and di-substituted amino wherein the substituents are independently selected from (C₁-C₆)alkyl; [(C₁-C₆)alkyl]-C(═O)—; [(C₁-C₆)alkoxy]-C(═O)—; [(C₁-C₆)alkyl]-SO₂—; and four- to eight-membered heterocyclyl containing one to four hetero atoms independently selected from nitrogen, oxygen and sulfur, wherein said heterocyclyl is optionally substituted with one to three substituents independently selected from hydroxy, (C₁-C₆)alkyl, NH₂-C(O═)—, [(C₁-C₆)alkyl]-NH—C(═O)—, [(C₁-C₆)alkyl]₂-N—C(═O)—, and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or

R^{Y1} and R^{Y2} taken together with the carbon atom to which they are attached form spiropyrrolidinyl or spiropiperidinyl, both of which are optionally N-substituted with a substituent selected from (C₁-C₆)alkyl, (C₁-C₆)alkyl-C(═O)—, [(C₁-C₆)alkyl]-C(═O)—(C₁-C₆)alkyl and aryl-(C═O)— wherein aryl is selected from phenyl and naphthyl;

R^{Y3} is hydrogen;

R^{Y4} is selected from

hydroxy;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{4a} are independently selected from hydrogen, (C₁-C₆)alkyl, [(C₁-C₈)alkyl]-C(═O)—, [(C₁-C₆)alkoxy]-C(═O)— and [(C₁-C₆)alkyl]-SO₂—; and

(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

R^{Y5}, R^{Y6} and R^{Y7} are independently selected from

hydrogen;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{2a}, R^{3a} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;

hetrocyclyl-(CH₂)ₙ₆— wherein n6 is an integer selected from 0, 1, 2, 3 and 4 and said heterocyclyl is four to eight membered containing one to three hetero atoms independently selected from nitrogen, oxygen and sulfur, wherein said heterocyclyl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and

hetroaryl-(CH₂)ₙ₇— wherein n7 is an integer selected from 0, 1, 2, 3 and 4 and said heteroaryl is five to ten membered containing one to three hetero atoms independently selected from nitrogen, oxygen and sulfur, wherein said heteroaryl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; or

R^{Y6} and R^{Y7} taken together with the nitrogen atom to which they are attached form a four to eight heterocyclyl optionally containing, in addition to the nitrogen atom, one to two additional hetero atoms independently selected from nitrogen, oxygen and sulfur, and said heterocyclyl is optionally substituted with one substituent selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—;

said A is optionally substituted in the fused benzene rings with one to four substituents independently selected from

halo;

hydroxy;

mercapto;

phenyl;

(C₁-C₆)alkyl optionally substituted with one to three substituents independently selected from halo, hydroxy, carboxy, (C₁-C₆)alkoxy, (C₁-C₆)alkoxy-C(═O)— and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂; and

(C₁-C₆)alkoxy optionally substituted with one to three substituents independently selected from halo, hydroxy, carboxy, (C₁-C₆)alkoxy, (C₁-C₆)alkoxy-C(═O)— and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and

Z is selected from

C(═O);

(CH₂)ₙ₈ wherein n8 is an integer selected from 0, 1 and 2; and

CHR^{Z1} wherein

R^{Z1} is selected from

carboxy;

(C₁-C₆)alkoxy-C(═O)—;

non-, mono- and di-substituted amino wherein the substituents are independently selected from (C₁-C₆)alkyl, [(C₁-C₆)alkyl]-C(═O)—, [(C₁-C₆)alkyl]-C(═O)—O— and [(C₁-C₆)alkyl]-SO₂—;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a1}R^{a2}N—C(═O)—, wherein R^{a1}, R^{a2}, R^{a3} and R^{4a} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

[C(═O)—NR^{Z11}R^{Z12}] wherein R^{Z11} and R^{Z12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—.

A further preferred class of compound of formula (I) of this invention is that wherein,

all R¹ are hydrogen

each R²is independently selected from hydrogen and halo;

X¹ is selected from (CH₂)ₙ₁ wherein n1 is an integer selected from 1, 2 and 3; O; NH; S; C(═O); SO₂; and N[(C₁-C₄)alkyl];

X² is selected from CH₂; O; NH; S; C(═O); SO₂; and N[(C₁-C₄)alkyl]; or

X¹ and X² taken together form CH═CH;

W¹ and W² are both CH₂;

A is AB wherein

Y^{b} is CR^{Y3}[C(═O)NR^{Y6}R^{Y7}]; and

Y^{c} is selected from

CR^{Y1}R^{Y2};

CR^{Y3}[C(═O)R^{Y4}];

CR^{Y3}[C(═O)NR^{Y6}R^{Y7}]; and

CR^{Y3}[NR^{Y6}R^{Y7}]; wherein

R^{Y1} and R^{Y2} are independently selected from

hydrogen;

hydroxy;

non-, mono- and di-substituted amino wherein the substituents are independently selected from (C₁-C₆)alkyl; [(C₁-C₆)alkyl]-C(═O)—; [(C₁-C₆)alkoxy]-C(═O)—; [(C₁-C₆)alkyl]—SO₂; and four- to eight-membered heterocyclyl containing one to four hetero atoms independently selected from nitrogen, oxygen and sulfur, wherein said heterocyclyl is optionally substituted with one to three substituents independently selected from hydroxy, (C₁-C₆)alkyl, NH₂—C(O═)—, [(C₁-C₆)alkyl]-NH—C(═O)—, [(C₁-C₆)alkyl]₂-N—C(═O)—, and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or

R^{Y1} and R^{Y2} taken together with the carbon atom to which they are attached form spiropyrrolidinyl or spiropiperidinyl, both of which are optionally N-substituted with a substituent selected from (C₁-C₆)alkyl, (C₁-C₆)alkyl-C(═O)—, [(C₁-C₆)alkyl]-C(═O)—(C₁-C₆)alkyl and aryl-(C═O)— wherein aryl is selected from phenyl and naphthyl;

R^{Y3} is hydrogen;

R^{Y4} is selected from

hydroxy;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

R^{Y5}, R^{Y6} and R^{Y7} are independently selected from

hydrogen;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;

hetrocyclyl-(CH₂)ₙ₆— wherein n6 is an integer selected from 0, 1, 2, 3 and 4 and said heterocyclyl is four to eight membered containing one to three hetero atoms independently selected from nitrogen, oxygen and sulfur, wherein said heterocyclyl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and

hetroaryl-(CH₂)ₙ₇— wherein n7 is an integer selected from 0, 1, 2, 3 and 4 and said heteroaryl is five to ten membered containing one to three hetero atoms independently selected from nitrogen, oxygen and sulfur, wherein said heteroaryl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; or

R^{Y6} and R^{Y7} taken together with the nitrogen atom to which they are attached form a four to eight heterocyclyl optionally containing, in addition to the nitrogen atom, one to two additional hetero atoms independently selected from nitrogen, oxygen and sulfur, and said heterocyclyl is optionally substituted with one substituent selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—;

said A is optionally substituted in the fused benzene rings with one to four substituents independently selected from

halo;

hydroxy;

mercapto;

phenyl;

(C₁-C₆)alkyl optionally substituted with one to three substituents independently selected from halo, hydroxy, carboxy, (C₁-C₆)alkoxy, (C₁-C₆)alkoxy-C(═O)— and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and

(C₁-C₆)alkoxy optionally substituted with one to three substituents independently selected from halo, hydroxy, carboxy, (C₁-C₆)alkoxy, (C₁-C₆)alkoxy-C(═O)— and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and

Z is C(═O).

Individual preferred compounds of this invention include

2,3-dihydro-1′-{3-[2-(N-methylaminocarbonyl)indolin-1-yl]-3-oxopropyl}spiro[1H-indene-1,4′-piperidine];

2,3-dihydro-1′-[3-(2-N,N-dimethylaminocarbonylindolin-1-yl)-3-oxopropyl]spiro[1H-indene-1,4′-piperidine];

2,3-dihydro-1′-[3-(2-morpholinocarbonylindolin-1-yl)-3-oxopropyl]spiro[1H-indene-1,4′-piperidine];

2,3-dihydro-1′-[3-(2-carbamoylindolin-1-yl)-3-oxopropyl]spiro[1H-indene-1,4′-piperidine]hydrochloride;

2,3-dihydro-1′-{3-[2-(1 -ethylprrolydin-3-yl)aminocarbonylindolin-1-yl]-3-oxopropyl}spiro[1H-indene-1,4′-piperidine];

2,3-dihydro-1′-{3-[2-(S)-(N,N-dimethylaminoethyl)aminocarbonylindolin-1-yl]-3-oxopropyl}spiro[1H-indene-1,4′-piperidine];

2,3-dihydro-1′-{3-[2-(S)-(2-hydroxyethyl)aminocarbonylindolin-1-yl]-3-oxopropyl}spiro[1H-indene-1,4′-piperidine];

2,3-dihydro-1′-{3-[2-(S)-(2-aminoethyl)aminocarbonylindolin-1-yl]-3-oxopropyl}spiro[1H-indene-1,4′-piperidine];

2,3-dihydro-1′-(3-[2-(S)-(2-acetamidoethyl)aminocarbonylindolin-1 -yl]-3-oxopropyl}spiro[1H-indene-1,4′-piperidine];

2,3-dihydro-1′-{3-[2-(S)-(2-methanesulfonamidoethyl)aminocarbonylindolin-1 -yl]-3-oxopropyl}spiro[1H-indene-1,4′-piperidine];

2,3-dihydro-1′-[3-(2-(S)-N-methylaminocarbonylindolin-1-yl)-3-oxopropyl]spiro[1H-indene-1,4′-piperidine];

2,3-dihydro-1′-[3-(2-(S)-N,N-dimethylaminocarbonylindolin-1-yl)-3-oxopropyl]spiro[1H-indene-1,4′-piperidine];

2,3-dihydro-1′-{3-[2-(S)-(4-morpholinecarbonyl)indolin-1 -yl]-3-oxopropyl}spiro[1H-indene-1,4′-piperidine]; and

2,3-dihydro-1′-[3-(2-(S)-aminocarbonylindolin-1-yl)-3-oxopropyl]spiro[l H-indene-1,4′-piperidine], or a salt thereof.

Another preferred class of compounds of formula (I) of this invention is that wherein

all R¹ are hydrogen

each R² is independently selected from hydrogen and halo;

X¹ is selected from (CH₂)ₙ₁ wherein n1 is an integer selected from 1, 2 and 3; O; NH; S; C(═O); SO₂; and N[(C₁-C₄)alkyl];

X² is selected from CH₂; O; NH; S; C(═O); SO₂; and N[(C₁-C₄)alkyl]; or

X¹ and X² taken together form CH═CH;

W¹ and W² are both CH₂;

A is AB wherein

Y^{b} is CR^{Y1}R^{Y2}; and

Y^{c} is selected from

CR^{Y1}R^{Y2} ;

CR^{Y3}[C(═O)R^{Y4}];

CR^{Y3}[C(═O)NR^{Y6}R^{Y7}]; and

CR^{Y3}[NR^{Y6}R^{Y7}]; or

Y^{b} and Y^{c} taken together form a group selected from CH₂-CH₂ and CH₂═CH₂;

R^{Y1} and R^{Y2} are independently selected from

hydrogen;

hydroxy;

non-, mono- and di-substituted amino wherein the substituents are independently selected from (C₁-C₆)alkyl; [(C₁-C₆)alkyl]-C(═O)—; [(C₁-C₆)alkoxy]-C(═O)—; [(C₁-C₆)alkyl]-SO₂—; and four- to eight-membered heterocyclyl containing one to four hetero atoms independently selected from nitrogen, oxygen and sulfur, wherein said heterocyclyl is optionally substituted with one to three substituents independently selected from hydroxy, (C₁-C₆)alkyl, NH₂—C(O═)—, [(C₁-C₆)alkyl]-NH—C(═O)—, [(C₁-C₆)alkyl]₂-N—C(═O)—, and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-Ca)alkoxy]-C(═O)— and [(C₁-C₆)alkyl]-SO₂—; and

(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or

R^{Y1} and R^{Y2} taken together with the carbon atom to which they are attached form spiropyrrolidinyl or spiropiperidinyl, both of which are optionally N-substituted with a substituent selected from (C₁-C₆)alkyl, (C₁-C₆)alkyl-C(═O)—, [(C₁-C₆)alkyl]-C(═O)—(C₁-C₆)alkyl and aryl-(C═O)— wherein aryl is selected from phenyl and naphthyl;

R^{Y3} is hydrogen;

R^{Y4} is selected from

hydroxy;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

R^{Y6} and R^{Y7} are independently selected from

hydrogen;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;

hetrocyclyl-(CH₂)ₙ₆— wherein n6 is an integer selected from 0, 1, 2, 3 and 4 and said heterocyclyl is four to eight membered containing one to three hetero atoms independently selected from nitrogen, oxygen and sulfur, wherein said heterocyclyl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and

hetroaryl-(CH₂)ₙ₇— wherein n7 is an integer selected from 0, 1, 2, 3 and 4 and said heteroaryl is five to ten membered containing one to three hetero atoms independently selected from nitrogen, oxygen and sulfur, wherein said heteroaryl is optionally substituted with one to three substituents independently selected from hydroxy; (C₁-C₆)alkyl; NH₂-C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; or

R^{Y6} and R^{Y7} taken together with the nitrogen atom to which they are attached form a four to eight heterocyclyl optionally containing, in addition to the nitrogen atom, one to two additional hetero atoms independently selected from nitrogen, oxygen and sulfur, and said heterocyclyl is optionally substituted with one substituent selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxyl-C(═O)— and [(C₁-C₆)alkyl]-SO₂—;

said A is optionally substituted in the fused benzene rings with one to four substituents independently selected from

halo;

hydroxy;

mercapto;

phenyl;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

Z is C(═O).

Individual preferred compounds of this invention include

2,3-dihydro-1′-[3-(2-methoxycarbonylindolin-1-yl)-3-oxopropyl]spiro[1H-indene-1,4′-piperidine];

2,3-dihydro-1′-[3-(indolin-1-yl)-3-oxopropyl]spiro[1H-indene-1,4′-piperidine];

2,3-dihydro-1′-[3-(2-(S)-methoxycarbonylindolin-1-yl)-3-oxopropyl]spiro[1H-indene-1,4′-piperidine];

2,3-dihydro-1′-indolyl-3-oxopropylspiro[1H-indene-1,4′-piperidine];

2,3-dihydro-1′-[3-(2-hydroxymethylindolin-1-yl)-3-oxopropyl]spiro[1H-indene-1,4′-piperidine]; and

2,3-dihydro-1′-[3-(2-methoxymethylindolin-1-yl)-3-oxopropyl]spiro[1H-indene-1,4′-piperidine], or a salt thereof.

Another preferred class of compound of formula (I) is that wherein

all R¹ are hydrogen

each R² is independently selected from hydrogen and halo;

X¹ is selected from (CH₂)ₙ₁ wherein n1 is an integer selected from 1, 2 and 3; O; NH; S; C(═O); SO₂; and N[(C₁-C₄)alkyl];

X² is selected from CH₂; C; NH; S; C(═O); SO₂; and N[(C₁-C₄)alkyl]; or

X¹ and X² taken together form CH═CH;

W¹ and W² are both CH₂;

A is AB wherein

Y^{b} is selected from

C(═O);

CR^{Y1}R^{Y2};

CR^{Y3}[C(═O)R^{Y4};

CR^{Y3}[NR^{Y5}C(═O)R^{Y4}];

CR^{Y3}[C(═O)NR^{Y6}R^{Y7}]; and

Y^{c} is selected from

O;

S;

SO₂;

NH;

N[(C₁-C₆)alkyl] wherein said (C₁-C₆)alkyl is optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;

N—(CH₂)ₙ₃-heterocyclyl wherein n3 is an integer selected from 0, 1, 2 and 3, and said heterocyclyl contains from four to eight ring atoms one or two of which are independently selected from nitrogen, oxygen and sulfur;

N—(CH₂)ₙ₄-aryl wherein n4 is an integer selected from 0, 1, 2 and 3, and said aryl is selected from phenyl and naphthyl; and

N—(CH₂)ₙ₅-heteroaryl wherein n5 is an integer selected from 0, 1, 2 and 3, and said heteroaryl is a five to ten membered aromatic heterocyclyl containing from one to four hetero atoms independently selected from nitrogen, oxygen and sulfur; wherein

R^{Y1} and R^{Y2} are independently selected from

hydrogen;

hydroxy;

non-, mono- and di-substituted amino wherein the substituents are independently selected from (C₁-C₆)alkyl; [(C₁-C₆)alkyl]-C(═O)—; [(C₁-C₆)alkoxy]-C(═O)—; [(C₁-C₆)alkyl]-SO₂—; and four- to eight-membered heterocyclyl containing one to four hetero atoms independently selected from nitrogen, oxygen and sulfur, wherein said heterocyclyl is optionally substituted with one to three substituents independently selected from hydroxy, (C₁-C₆)alkyl, NH₂—C(O═)—, [(C₁-C₆)alkyl]-NH—C(═O)—, [(C₁-C₆)alkyl]₂-N—C(═O)—, and non-, mono- and di-substituted amino wherein the substituents are independently selected from (C₁-C₆)alkyl, [(C₁-C₈)alkyl]-C(═O)—, [(C₁-C₆)alkoxy]-C(═O)— and [(C₁-C₆)alkyl]-SO₂—;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or

R^{Y1} and R^{Y2} taken together with the carbon atom to which they are attached form spiropyrrolidinyl or spiropiperidinyl, both of which are optionally N-substituted with a substituent selected from (C₁-C₆)alkyl, (C₁-C₆)alkyl-C(═O)—, [(C₁-C₆)alkyl]-C(═O)—(C₁-C₆)alkyl and aryl-(C═O)— wherein aryl is selected from phenyl and naphthyl;

R^{Y3} is hydrogen;

R^{Y4} is selected from

hydroxy;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

R^{Y5}, R^{Y6} and R^{Y7} are independently selected from

hydrogen;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;

hetrocyclyl-(CH₂)ₙ₆— wherein n6 is an integer selected from 0, 1, 2, 3 and 4 and said heterocyclyl is four to eight membered containing one to three hetero atoms independently selected from nitrogen, oxygen and sulfur, wherein said heterocyclyl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and

hetroaryl-(CH₂)ₙ₇— wherein n7 is an integer selected from 0, 1, 2, 3 and 4 and said heteroaryl is five to ten membered containing one to three hetero atoms independently selected from nitrogen, oxygen and sulfur, wherein said heteroaryl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; or

R^{Y6} and R^{Y7} taken together with the nitrogen atom to which they are attached form a four to eight heterocyclyl optionally containing, in addition to the nitrogen atom, one to two additional hetero atoms independently selected from nitrogen, oxygen and sulfur, and said heterocyclyl is optionally substituted with one substituent selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—;

said A is optionally substituted in the fused benzene rings with one to four substituents independently selected from

halo;

hydroxy;

mercapto;

phenyl;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

Z is selected from

C(═O);

(CH₂)ₙ₈ wherein n8 is an integer selected from 0, 1 and 2; and

CHR^{Z1} wherein

R^{Z1} is selected from

carboxy;

(C₁-C₆)alkoxy-C(═O)—;

non-, mono- and di-substituted amino wherein the substituents are independently selected from (C₁-C₆)alkyl, [(C₁-C₆)alkyl]-C(═O)—, [(C₁-C₆)alkyl]-C(═O)—O— and [(C₁-C₆)alkyl]-SO₂—;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxyl-C(═O)— and [(C₁-C₆)alkyl]-SO₂—; and

[C(═O)—NR^{Z11}R^{Z12}] wherein R^{Z11} and R^{Z12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—.

Individual preferred compounds of this invention include

2,3-dihydro-1′-[3-(benzimidazol-2-one-1-yl)propyl]spiro[1 H-indene-1,4′-piperidine];

2,3-dihydro-1′-[3-(benzothiazol-2-one-1-yl)propyl]spiro[1 H-indene-1,4′-piperidine];

2,3-dihydro-1′-[3-(2-oxo-1,3-benzoxazol-3(2H)-yl)propyl]spiro[1H-indene-1,4′-piperidine];

2,3-dihydro-1′-[3-(2-hydroxymethylbenzimidazol-1-yl)-3-oxopropyl]spiro[1H-indene-1,4′-piperidine];

2,3-dihydro-1′-[3-(3-ethylbenzimidazol-2-one-1 -yl)propyl]spiro[1H-indene-1,4′-piperidine];

2,3-dihydro-1′-[3-(2-acetamidobenzimidazol-1-yl)propyl]spiro[1H-indene-1,4′-piperidine];

2,3-dihydro-1′-{3-[3-(2-hydroxyethyl)benzimidazol-2-one-1-yl)propyl)spiro[1H-indene-1,4′-piperidine];

2,3-dihydro-1′-{3-[3-(2-aminoethyl)benzimidazol-2-one-1-yl)propyl}spiro[1H-indene-1,4′-piperidine]; and

2,3-dihydro-1′-{3-[3-(2-acetamidoethyl)benzimidazol-2-one-1-yl)propyl}spiro[1H-indene-1,4′-piperidine], or a salt thereof.

Another preferred class of compound of formula (I) of this invention is that wherein

all R¹ are hydrogen

each R² is independently selected from hydrogen and halo;

X¹ is selected from (CH₂)ₙ₁ wherein n1 is an integer selected from 1, 2 and 3; O; NH; S; C(═O); SO₂; and N[(C₁-C₄)alkyl];

X² is selected from CH₂; O; NH; S; C(═O); SO₂; and N[(C₁-C₄)alkyl]; or

X¹ and X² taken together form CH═CH;

W¹ and W² are independently selected from CR^{W1}R^{W2},

wherein

R^{W1} and R^{W2} are independently selected from

hydrogen;

halo;

hydroxy;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;

(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7} R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;

C(═O)—[(C₁-C₆)alkyl] wherein said (C₁-C₆)alkyl is optionally substituted with one to three substituents independently selected from halo, hydroxy, carboxy, [(C₁-C₆)alkyl]-C(═O)—, (C₁-C₅)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;

C(═O)—NR^{W11}R^{W12} wherein R^{W11} and R^{W12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]

NR^{W13}R^{W14} wherein R^{W13} and R^{W14} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂

aryl selected from phenyl and naphthyl; and

four- to eight-membered heterocyclyl containing one to four hetero atoms independently selected from nitrogen, oxygen and sulfur;

A is AC wherein

Y^{d}, Y^{e} and Y⁴ are independently selected from

C(═O)

CR^{Y1}R^{Y2};

CR^{Y3}[C(═O)R^{Y4}];

CR^{Y3}[NR^{Y5}C(═O)R^{Y4}];

CR^{Y3}[C(═O)NR^{Y6}R^{Y7}];

CR^{Y3}[NR^{Y6}R^{Y7}];

O;

S;

SO₂;

NH;

N[(C₁-C₆)alkyl] wherein said (C₁-C₆)alkyl is optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{, R}^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;

N—(CH₂)ₙ₃-heterocyclyl wherein n3 is an integer selected from 0, 1, 2 and 3, and said heterocyclyl contains from four to eight ring atoms one or two of which are independently selected from nitrogen, oxygen and sulfur;

N—(CH₂)ₙ₄-aryl wherein n4 is an integer selected from 0, 1, 2 and 3, and said aryl is selected from phenyl and naphthyl; and

N—(CH₂)ₙ₅-heteroaryl wherein n5 is an integer selected from 0, 1, 2 and 3, and said heteroaryl is a five to ten membered aromatic heterocyclyl containing from one to four hetero atoms independently selected from nitrogen, oxygen and sulfur;

R^{Y1} and R^{Y2} are independently selected from

hydrogen;

hydroxy;

non-, mono- and di-substituted amino wherein the substituents are independently selected from (C₁-C₆)alkyl; [(C₁-C₆)alkyl]-C(═O)—; [(C₁-C₆)alkoxy]-C(═O)—; [(C₁-C₆)alkyl]-SO₂—; and four- to eight-membered heterocyclyl containing one to four hetero atoms independently selected from nitrogen, oxygen and sulfur, wherein said heterocyclyl is optionally substituted with one to three substituents independently selected from hydroxy, (C₁-C₆)alkyl, NH₂—C(O═)—, [(C₁-C₆)alkyl]-NH—C(═O)—, [(C₁-C₆)alkyl]₂-N—C(═O)—, and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or

R^{Y1} and R^{Y2} taken together with the carbon atom to which they are attached form spiropyrrolidinyl or spiropiperidinyl, both of which are optionally N-substituted with a substituent selected from (C₁-C₆)alkyl, (C₁-C₆)alkyl-C(═O)—, [(C₁-C₆)alkyl]-C(═O)—(C₁-C₆)alkyl and aryl-(C═O)— wherein aryl is selected from phenyl and naphthyl;

R^{Y3} is hydrogen;

R^{Y4} is selected from

hydroxy;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)− and (C₁-C₆)alkyl]-SO₂—; and

(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

R^{Y5}, R^{Y6} and R^{Y7} are independently selected from

hydrogen;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;

hetrocyclyl-(CH₂)ₙ₆— wherein n6 is an integer selected from 0, 1, 2, 3 and 4 and said heterocyclyl is four to eight membered containing one to three hetero atoms independently selected from nitrogen, oxygen and sulfur, wherein said heterocyclyl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and

hetroaryl-(CH₂)ₙ₇— wherein n7 is an integer selected from 0, 1, 2, 3 and 4 and said heteroaryl is five to ten membered containing one to three hetero atoms independently selected from nitrogen, oxygen and sulfur, wherein said heteroaryl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; or R^{Y6} and R^{Y7} taken together with the nitrogen atom to which they are attached form a four to eight heterocyclyl optionally containing, in addition to the nitrogen atom, one to two additional hetero atoms independently selected from nitrogen, oxygen and sulfur, and said heterocyclyl is optionally substituted with one substituent selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and

said A is optionally substituted in the fused benzene rings with one to four substituents independently selected from

halo;

hydroxy;

mercapto;

phenyl;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

Z is selected from

C(═O);

(CH₂)ₙ₈ wherein n8 is an integer selected from 0, 1 and 2; and

CHR^{Z1} wherein

R^{Z1} is selected from

carboxy;

(C₁-C₆)alkoxy-C(═O)—;

non-, mono- and di-substituted amino wherein the substituents are independently selected from (C₁-C₆)alkyl, [(C₁-C₆)alkyl]-C(═O)—, [(C₁-C₆)alkyl]-C(═O)—O and [(C₁-C₆)alkyl]-SO₂—;

(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and

[C(═O)—NR^{Z11}R^{Z12}] wherein R^{Z11} and R^{Z12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C6)alkoxy]-C(═O)— and [(C₁-C₆)alkyl]-SO₂.

Individual preferred compounds of this invention include 2,3-dihydro-140 -[3-(2-oxo-3,4-dihydro-1 (2H)-quinolinyl)propyl]spiro[1H-indene-1,4′-piperidine] and 2,3-dihydro-1′-[3-methyl-2-oxo-3,4-dihydro-1(2H)-quinazolinyl)propyl]spiro[1H-indene-1,4′-piperidine ]; or a salt thereof.

Accordingly, this invention relates to a pharmaceutical composition comprising an effective amount of a compound of formula I defined as above and a pharmaceutically acceptable carrier for treating a disease or medical condition mediated by ORL1-receprot and its endogeneous ligand in a mammal including a human.

A preferred pharmaceutical composition of this invention comprises a compound of formula I defined as above having selectivity for ORL-1 receptor.

A further preferred pharmaceutical composition of this invention comprises a compound of formula I defined as above having antagonist effect for ORL-1 receptor.

A further preferred pharmaceutical composition of this invention comprises a compound of formula I defined as above which is a selective antagonist for ORL-1 receptor.

Therefore, a pharmaceutical composition of this invention comprising a compound of formula I defined as above is useful for treating or preventing a disease or medical condition selected from pain; eating disorders including anorexia and bulimia; anxiety and stress conditions; immune system diseases; locomotor disorder; eating disorder; memory loss, cognitive disorders and dementia including senile dementia and those diseases caused by Alzheimer's disease, Perkinson's disease or other neurodegenerative pathologies; epilepsy or convulsion and symptoms associated therewith; a central nervous system disorder related to gulutamate release action, anti-epileotic action, disruption of spatial memory, serotonin release, anxiolytic action, mesolimbic dopaminergic transmission, rewarding propaerties of drug of abuse, modulation of striatal and glutamate effects on locomotor activity; cardiovascular disorders hypotension, bradycardia and stroke; renal disorders including water excretion, sodium ion excretion and syndrome of inappropriate secretion of antidiuretic hormone (SIADH); gastrointestinal disoders; airway disorders including adult respiratory distress syndrome (ARDS); autonomic disorders including suppression of micturition reflex; metabolic disorders including obesity; cirrhosis with ascites; sexsual dysfunctions; and altered pulmonary function including obstructive pulmonary disease.

This invention also relates to a method for treating or preventing a disease or condition in a mammal including a human, which disease or condition is mediated by ORL-1 receptor and its endogeneous ligand, comprising administering an effective amount of a compound of formula I defined as above to a mammal including a human, which suffered from such disease or condition.

More specifically, this invention relates to a method for treating or preventing the aforementioned disease or medical condition, wherein said compound has selectivity for ORL-1 receptor.

More specifically, this invention relates to a method of treating or preventing the aforementioned disease or medical condition, wherein said compound has antagonist effect for ORL-1 receptor.

More specifically, this invention relates to a method for treating or preventing the aforementioned disease or medical condition, wherein said compound is a selective antagonist for ORL-1 receptor.

Accordingly, this invention relates to a method for treating or preventing the aforementioned disease or medical condition wherein said disease or condition is selected from pain; eating disorders including anorexia and bulimia; anxiety and stress conditions; immune system diseases; locomotor disorder; eating disorder; memory loss, cognitive disorders and dementia including senile dementia and those diseases caused by Alzheimer's disease, Perkinson's disease or other neurodegenerative pathologies; epilepsy or convulsion and symptoms associated therewith; a central nervous system disorder related to gulutamate release action, anti-epileotic action, disruption of spatial memory, serotonin release, anxiolytic action, mesolimbic dopaminergic transmission, rewarding propaerties of drug of abuse, modulation of striatal and glutamate effects on locomotor activity; cardiovascular disorders hypotension, bradycardia and stroke; renal disorders including water excretion, sodium ion excretion and syndrome of inappropriate secretion of antidiuretic hormone (SIADH); gastrointestinal disoders; airway disorders including adult respiratory distress syndrome (ARDS); autonomic disorders including suppression of micturition reflex; metabolic disorders including obesity; cirrhosis with ascites; sexsual dysfunctions; and altered pulmonary function including obstructive pulmonary disease.

### General Synthesis

The compounds of formula I of the present invention may be prepared according to known preparation methods, or General Procedures or preparation methods illustrated in the following reaction Schemes. Unless otherwise indicated R¹, R², X¹, X², W¹, W², A and Z, and groups or substituents thereof, in the reaction Schemes and discussion that follow are defined as above. Unless otherwise indicated, reactions in this specification may be carried out at about ambient pressure (i.e., 760 mmHg) and about room temperature (i.e., 25° C.).

Typical preparation procedures for compounds of formula I of the present invention are as follow:

Protecting Groups:

Amino, hydroxy, mercapto or the like may be protected with a protecting group, and the protecting group may be subsequently removed in an appropriate reaction step according to a known procedure (e.g., Protective Groups in Organic Synthesis edited by T. W. Greene et al. (John Wiely & Sons, 1991)). For example, a primary or a secondary amine may be typically protected by reaction with benzyl chloride in K₂CO₃ solution, and the benzyl group (abbreviated as Bn) may be removed by catalytic hydrogenation over palladium-carbon. Introduction for t-butoxycarbonyl (abbreviated as Boc) to amino group may be carried out using (BOC)₂O under basic condition, and the protecting group may be removed in HCl/EtOAc. Hydroxy may protected with t-butyldimethylsilyl (abbreviated as TBS or TBDMS) in alkylation using NaH. The protecting group may be introduced with TBDMSCI in imidazole and DMF and removed using an appropriate reagent such as tetrabutylammonium fluoride.

Leaving Groups/Introductions of Sulfonyl Groups:

Leaving group used in a reaction described hereafter are known to those skilled in the art. These leaving groups include halo such as Cl, Br and I; sulfonic esters such as TfO (triflates), MsO (mesylates), TsO (tosylates); and the like. These groups may be introduced to an appropriate compound according to methods known to those skilled in the art (e.g., (a) halogenation using triphenylphosphine/CX₄ wherein X is halo (PPh₃/CX₄); (b) reaction with TsCl; and (c) reaction with MsCl).

Halogenations:

Carboxylic acids or alcohols may be converted to alkyl or acyl halides using halogenation reagents. Conversions of alcohols or carboxylic acids respectively to alkyl halides or acyl halides may be typically carried out using SOCl₂, PCl₅, PCl₃, POCl₃, HBr, PBr₃, HI or the like.

Alkylations:

Alkylations may be carried out according to a procedure known to those skilled in the art. More specifically, a primary or secondary amine may be alkylated to a secondary or tertialy amine with a halo alkyl (preferably as a bromide or iodide compound) in the presence of an alkali metal ion such as potassium ion, base or a mixture thereof. This alkylation may be also carried out using a nucleophilic strong base that serves to remove the proton of the secondary amine radical. Instead of halides, sulfates or sulfonates may be used in these reactions. Alkylations of alcohols may be carried out using diazo compounds preferably in the presence of a catalyst such as fluoboric acid (HBF₄) or silica gel. For the alkylations, suitable solvents include polar aprotic solvents such as dimethylformamide (DMF), dimethylsulfoxide, acetonitrile (MeCN), acetone, sulfur dioxide, dichloromethane, hexane and the like; and protic solvents such as water, alcohols such as methanol (MeOH) and ethanol (EtOH), ethylene glycol and the like, or a combination thereof. These reactions may be typically carried out at a temperature from about 0° C. to the reflux temperature of a solvent to be used for from about 1 minute to 30 hours.

Aminations:

Aminations of alkanols or alkyl halides may be carried out by reactions with cyclic imide compounds such as N-phthalimides followed by hydrazinolysis or hydrolysis. If required, the reactions with phthalimides may be carried out using organophosphorous reagents with or without azo compounds.

Amidations:

If appropriate, a base such as triethylamine, or a base catalysis such as N,N-dimethylaminopyridine (DMAP), 4-pyrrolidinopyridine (PPY) or the like may be employed in this reaction. Suitable solvents for this reaction include hexane, dichloromethane, tetrahydrofuran (THF), pyridine and the like.

Amidation-1—Acylayion of Amines by Acyl Halides:

Acyl halids may be treated with ammonia or amines for the preparation of amides. This reaction may be carried out and in the presence or absence of an aqueous alkali which may capture the liberated halide ion and controlled by cooling or dilution. Acyl halide may also be reacted with arylamines, hydrazine or hydroxylamine under the similar conditions. Amino protections using carbobenzoxy group (abbreviated as Cbz) or t-butoxycarbonyl group (abbreviated as Boc) may be carried out in this way.

Amidation 2—Acylation of Amines by Anhydride:

This reaction may be carried out with ammonia or primary or secondary amines according to a similar procedure described in Amidation 1 above. Ammonia and primary amines may give imides including cyclic imides, wherein two acyl groups are attached to the nitrogen.

Amidation 3—Acylation of Amines by Carboxylic Acids:

Carboxylic acids may be treated with ammonia or amine compounds to give amides. This amidation may be carried out in the presence of a coupling agent with or without an additional base at about room temperature. A coupling agent such as dicyclohexylcarbodiimide (DCC) used in a peptide synthesis may be applied to the amidations. Other suitable coupling agents used in these amidations include N,N′-carbonyldiimidazole (CDI), diisopropylcarbodiimide (DIPC), 1-ethyl-3-(3-dimethylaminopropyl)carbodiimide (WSC, water soluble carbodiimide), benzotriazole-1-yloxy-tris(dimethylamino)phosphonium hexafluorophosphate (BOP) and diphenylphosphorylazide (DPPA) and the like. A cyclic amine may be acylated according to a method analogous to these amidations. If amines are subjected to this reaction in its halogen salt forms, additional amines may be used for trapping hydrogen halides formed.

Amidation 4—Acylation of Amines by Carboxylic Esters:

Carboxylic esters may be converted to unsubstituted, N-substituted or N,N-disubstituted amides. This reaction may be carried out in the presence of a strong base catalysis as well as catalysis by cyanide ion under a high pressure. Hydrazides and hydroxamic acids may be prepraed from carboxylic esters with hydrazine and hydroxylamine respectively under similar reaction conditions.

Amidation 5—Acylation of Amines by Amides or Other Acid Derivatives:

A salt of an amine may be subjected to this reaction. In this reaction, NH₂ usually acts as a leaving group. Secondary and primary amines (in the form of their salts) are the most common reagents in this reaction. Acid derivatives, which may be converted to amides, include thiol acids, thiol ethers, acyloxyboranes, 1,1,1-trihalo ketones, α-keto nitrils, acyl azides and the like.

These amidations may be carried out in a reaction inert solvent such as dichloromethane (CH₂Cl₂), alcohols such as methanol, ethanol or buthanol (BtOH), acetonitrile, tetrahydrofuran (THF), dimethyfuran (DMF), or pyridine or a combination thereof, at a temperature from about 0° C. to the reflux temperature of a solvent, for from about 5 minutes to 48 hours.

Hydrolysis of Esters:

Hydrolysis of esters may be carried out in the presence of an acid, base, metal ion, enzyme or nucleophile according to a method known to those skilled in the art. The hydrolysis of esters may be carried out in a reaction inert solvent at a temperature from about 0° C. to the reflux temperature of the solvent for from about 1 to 24 hours. Suitable solvents for the reactions include alcohols such as methanol, ethanol, tetrahydrofuran, acetic acid and the like.

Esterifications:

Carboxylic acids and alcohols afford esters using acid catalysis. Typical catalysis for this reaction include conc. HCl, anhydrous sulfuric acid, p-toluenesulfonic acid and the like. The alcohol generally servers as the solvent, but other reaction inert solvent such as toluene or xylene may be used. The alcohol may be used in large excess, and the water from the reaction mixture may be removed.

Reductions:

Reductions may be carried out using reducing agents. Typical reducing agents are lithium aluminum hydride, lithium triethylborohydride (LiEt₃BH), a complex formed from lithium trimethoxyaluminum hydride (LiAIH(OMe)₃) and Cul. Typical milder reducing agents are NaBH₄ and the like in a dipoler aprotic solvent such as Me₂SO, DMF or sulfolane. Other reducing agents are zinc with acid or base, SnCl₂, chromium(II) ion and the like. For example, carboxylic acids may be reduced to primary alcohols by LiAlH₄ at about room temperature, and nitro group may be reduced to amino group by reaction with zinc.

Schemes 1-1, 1-2 and 1-3 illustrate embodiments of preparation process for a compound of formula (I).

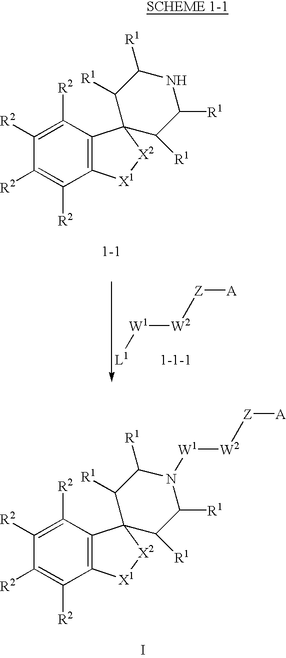

Scheme 1-1 illustrates a preparation method of a compound of formula I of the present invention. This method comprises alkylation of a spiro-piperidine compound of formula 1-1 by a compound of formula 1-1-1 wherein L¹ is a leaving group. This reaction may be carried out according to an alkylation of an amine compound. In a preferred embodiment of this reaction, a compound of formula 1-1 may be used as potassium salt, then reacted with a compound of formula 1-1-1 wherein the leaving group L¹ may be halo. The potassium salt of a compound formula 1-1 may be prepared by treating said compound with a potassium salt such as potassium carbonate, potassium hydroxide or a combination thereof. The following alkylation may be carried out at an elevated temperature, for example at about the reflux temperature of a reaction inert solvent used. Typically, this reaction may be carried out in acetonitrile (MeCN) using potassium carbonate (K₂CO₃) and potassium iodide (KI).

Scheme 1-2 illustrates another preparation method of a compound of formula (I).

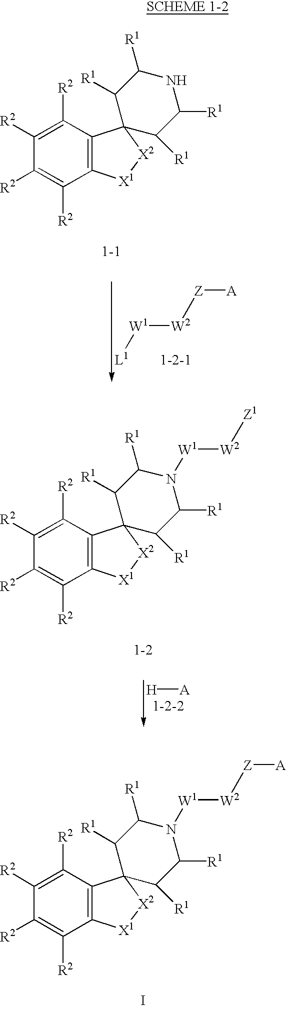

A compound of formula I may be prepared from a compound of formula 1-1 by alkylation with a compound of formula 1-2-1 followed by an amination with a compound of formula 1-2-2. In formula 1-2-1, Z¹is Z as defined in formula (I) or its analogous group comprising a leaving group, carbonyl, hydroxy or carboxy; and L¹is a leaving group similar to L¹ in formula 1-1-1 described in Scheme 1-1. Formula 1-2-2 means either of formulae AA-H, AB-H and AC-H as described below.

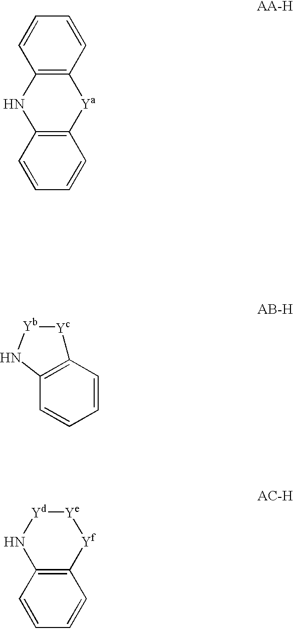

Namely, these compounds are reduced forms of substituent represented by “A” in formula (I) in this specification.

Alkylation of a compound of formula 1-1 with a compound of formula 1-2-1 may be carried out under similar conditions described in Scheme 1-1 in this specification to afford a compound of formula 1-2.

Then, the compound of formula 1-2 thus obtained may be reacted with a compound of formula 1-2-2. A compound of formula 1-2 wherein Z¹ comprises a leaving group may be coupled with a compound of formula 1-2-2 by alkyklation under similar reaction conditions as described in Scheme 1-1 or 1-2 in this specification. A compound of formula 1-2 wherein Z¹ comprises carboxy may be coupled with a compound of formula 1-2-2 by amidation by a peptide formation known to those skilled in the art.

A compound of formula I of the present application wherein A is AB as defined above may be also prepared according to a preparation method described in Scheme 1-3.

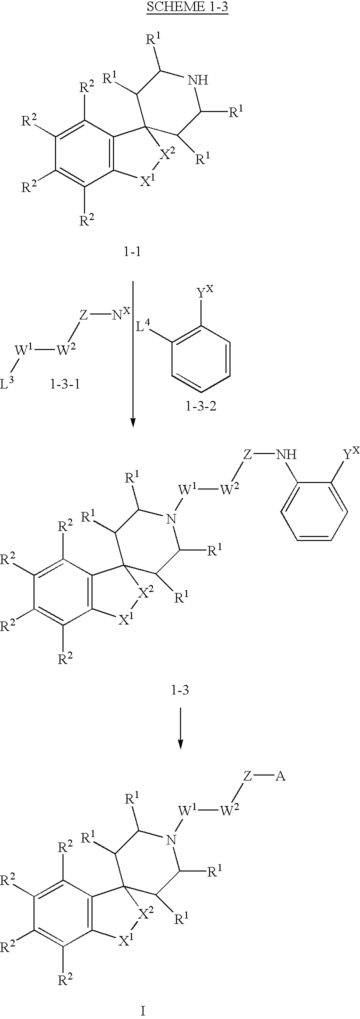

Preparation processes in Scheme 1-3 is preferably useful for compounds of formula I wherein in A is an optionally substituted benzofuzed heteroaryl ring containing a nitrogen atom and additional hetero atoms. A typical benzofuzed ring in the compounds is benzimidazolyl, benzothiazolyl or benzoxazolyl ring.

As shown in Scheme 1-3 the preparation process comprises:

Step 1—reaction between compounds of formula 1-1 may be reacted with compounds of formula 1-3-1, wherein L³ is a leaving group such as halo and N^{x} is amino, phthalimido or the like;

Step 2—reaction between compounds obtained in Step 1 with compounds of formula 1-3-2 to give compounds of formula 1-3; and

Step 3—cyclization of compounds of formula 1-3 to yield compounds of formula 1.

The reactions in Step 1 and 2 are alkylations of amine compounds. These reactions may be typically carried out in the presence of potassium ion. Resulting compounds in Step 1 wherein N^{x} is phthalimido may be converted to amine by deprotection with hydrazine prior to Step 2. The reaction in Step 3 may be carried out using carboxylic acids optionally in the presence of acid or a cyano halide.

The subject invention also includes isotopically-labelled compounds, which are identical to those recited in formula (I), but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assay. Tritiated, i.e., ³H, and carbon-14, i.e., ¹⁴C, isotopes are particularly preferred for their ease of presentation and detectability. Further, substitution with heavier isotopes such as deutrium, i.e., ²H, can afford therapeutic advantage resulting from greater metabolic stability, for example increased in vivo half-life or reduced dosage requirement and, hence, may be preferred in some circumstances. Isotopically labelled compounds of formula (I) of this invention and prodrugs thereof can generally be prepared by carrying out the procedure disclosed in above-disclosed Schemes and/or Examples and Preparations below, by submitting a readily available isotopically labelled reagent for a non-isotopically labelld reagent.

The compounds of Formula (I) of this invention are basic, therefore they will form acid-addition salts. All such salts are within the scope of this invention. However, it is necessary to use an acid addition salt which is pharmaceutically-acceptable for administration to a mammal. The acid-addition salts can be prepared by standard methods. For example, the salts may be prepared by contacting the basic compounds with acid in substantially equivalent proportions in water or an organic solvent such as methanol or ethanol, or a mixture thereof. The salts can be isolated by crystallization from or evaporation of the solvent. Typical salts which can be formed are the hydrochloride, nitrate, sulfate, bisulfate, phosphate, acetate, lactate, citrate, tartrate, succinate, maleate, fumarate, gluconate, saccharate, benzoate, methanesulfonate, ethanesulfonate, benzenesulfonate, p-toluenesulfonate, oxalate and pamoate (1,1′-methylene-bis-(2-hydroxy-3-naphtoate)) salts.

In addition, when the compounds of this invention form hydrates or solvates they are also within the scope of this invention.

The compounds of Formula (I) have been found to possess selective affinity for ORL1-receptors and ORL-1 receptor antagonist activity. Thus, these compounds are useful as an analgesic, anti-inflammatory, diuretic, anesthetic, neuroprotective, anti-hypertensive and anti-anxiety agent, and the like, in mammalian subjects, especially humans in need of such agents. The affinity, antagonist activities and analgesic activity can be demonstrated by the following tests respectively.

Selective Affinity for ORL1 -receptors:

ORL1 -Receptor Binding Assay:

The human ORL1 receptor transfected HEK-293 cell membranes were incubated for 45 min at 22° C. with 0.4 nM [³H]nociceptin, 1.0 mg of wheat germ agglutinin-coated SPA beads and various concentrations of test compounds in a final volume of 200 μl of 50 mM HEPES buffer pH 7.4 containing 10 mM MgCl₂ and 1 mM EDTA. Non-specific binding was determined by the addition of 1 μM unlabeled nociceptin. After the reaction, the assay plate was centrifuged at 1,000 rpm for 1 min and then the radioactivity was measured by a Liquid Scintillation Counter.

μ-Receptor Binding Assay:

The human Mu receptor transfected CHO-K1 cell membranes were incubated for 45 min at 220° C. with 1.0 nM [³H]DAMGO, 1.0 mg of wheat germ agglutinin-coated SPA beads and various concentrations of test compounds in a final volume of 200 μl of 50 mM Tris-HCl buffer pH 7.4 containing 5 mM MgCl₂. Non-specific binding was determined by the addition of 1 μM unlabeled DAMGO. After the reaction, the assay plate was centrifuged at 1,000 rpm for 1 min and then the radioactivity was measured by a Liquid Scintillation Counter.

Each percent non specific binding thus obtained is graphed as a function of compound concentration. A sigmoidal curve is used to determine 50% bindings (i.e., IC₅₀ values).

In this testing, the preferred compounds prepared in the working examples appearing hereafter demonstrated higher binding affinity for ORL1-receptors than for mu-receptors.

IC₅₀ (ORL1 -receptors) nM/IC₅₀ (mu-receptors) nM<1.0

ORL1 Receptor Functional Assay:

The human ORL1 receptor transfected HEK-293 cell membranes were incubated with 400 pM [³⁵S]GTPγS, 50 nM nociceptin and various concentrations of test compounds in assay buffer (20 mM HEPES, 100 mM NaCl, 5 mM MgCl₂, 1 mM EDTA, 5 mM GDP, 1 mM DTT, pH 7.4) containing 1.5mg of wheat germ agglutinin-coated SPA beads for 60 or 90 min at 25° C. in a final volume of 200 μl. Basal binding was assessed in the absence of nociceptin and non-specific binding was defined by the addition of unlabelled 10 mM GTPγS. Membrane-bound radioactivity was detected by a Liquid Scintillation Counter.

Analgesic Tests:

Tail Flick Test in Mice:

The latency time to withdrawal f the tail from radiant heat stimulation is recorded before and after administration of test compounds. Cut-off time is set to 8 sec.

Acetic Acid Writhing Test in Mice:

Acetic acid saline solution of 0.7% (v/v) is injected intraperitoneally (0.16 ml/10 g body weight) to mice. Test compounds are administered before acetic acid injection. As soon as acetic acid injection, animals are placed in a 1 liter beaker and writhing is recorded for 15 min.

Formalin Licking Test in Mice:

Formalin-induced hind paw licking is initiated by a 20 micro liters subcutaneous injection of a 2% formaline solution into a hind paw of mice. Test compounds are administered prior to formalin injection. Total licking time is recorded for 45 min after formalin injection.

Carrageenan-Induced Mechanical Hyperalgesia Test in Rats:

The response to mechanical nociceptive stimulus is measured using an algesiometer (Ugo Basile, Italy). The pressure is loaded to the paw until rats withdrawal the hind paw. Lambda-Carrageenan saline solution of 1% (w/v) is injected subcutaneously into the hind paw and the withdrawal response is measured before and after the injection. Test compounds are administered at appropriate time point.

Carrageenan-Induced Thermal Hyperalgesia Test in Rats:

The response to thermal nociceptive stimulus is measured using an plantar test apparatus (Ugo Basile, Italy). The radiant heat stimuli is applied to the paw until rats withdrawal the hind paw. Lambda-Carrageenan saline solution of 2% (w/v) is injected subcutaneously into the hind paw and the withdrawal response is measured before and after the injection. This testing method is described in K. Hargreaves, et al., Pain 32:77-88, 1988.

Chronic Contriction Injury Model (CCl Model):

Chronic contriction injury is made according to Bennett's method (Bennett, et al., Pain 83:169-182, 1999). Tactile allodynia in rats is assessed using the von Frey hairs (Stoelting, Ill.) before and after administration with test compounds.

The compounds of Formula (I) of this invention can be administered by conventional pharmaceutical practice via either the oral, parenteral or topical routes to mammals, for the treatment of the indicated diseases. For administration to human patient by either route, the dosage is in the range of about 0.01 mg/kg to about 3000mg/kg body weight of the patient per day, preferably about 0.01 mg/kg to about 1 000mg/kg body weight per day administered singly or as a divided dose. However, variations will necessarily occur depending upon the weight and condition of the subject being treated, compound employed, the disease state being treated and the particular route of administration chosen.

The compounds of the present invention may be administered alone or in combination with pharmaceutically acceptable carriers by either of the above routes previously indicated, and such administration can be carried out in single or multiple doses. Generally, the compounds can be combined with various pharmaceutically acceptable carriers in the form of tablets, powders, capsules, lozenges, trochees, hard candies, powders, sprays, creams, salves, suppositories, jellies, gels, pastes, lotions, ointments, suspensions, solutions, elixirs, syrups or the like. Such pharmaceutical carriers include solvents, excipients, coating agents, bases, binders, lubricants, disintegrants, solubilizing agents, suspending agents, emulsifing agents, stabilizers, buffering agents, tonicity agents, preservatives, flavorating agents, aromatics, coloring agents and the like.

For example, the tablets can contain various excipients such as starch, lactose, glucose, microcrystalline cellulose, calcium sulfate, calcium carbonate, talc, titanium oxide and the like, coating agents such as gelatin, hydroxypropylcellulose and the like, binding agents such as gelatin, gum arabic, methylcellulose and the like, and the disintegrating agents such as starch, agar, gelatine, sodium hydrogencarbonate and the like. Additionally, lubricating agents such as magnesium stearate and talc are often very useful for tabletting purposes. Solid compositions of a similar type may also be employed as fillers in gelatine capsules; preferred materials in this connection also include lactose as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with diluents such as water, ethanol, propylene glycol, glycerin and various like combinations thereof.

In general, the therapeutically-effective compounds of this invention are present in such oral dosage forms at concentration levels ranging 5% to 70% by weight, preferably 10% to 50% by weight.

The compounds of the present invention in the form of a solution may be injected parenterily such as intradermaly, subcutaneously, intravenously or intramuscularly. For example the solutions are sterile aqueous solutions, aqueous suspensions and an edible oil solutions. The aqueous solutions may be suitably buffered (preferably pH>8), and may contain enough salts or glucose to make the solution isotonic with blood. The aqueous solutions are suitable for intravenous injection purposes. The aqueous suspensions may contain a suitable dispersing or suspending agents such as sodium carboxymethylcellulose, methylcellulose, polyvinylpyrrolidone or gelatin. The aqueous suspensions can be used for subcutaneous or intramuscular injections. The edible oil such as cottonseed oil, sesame oil, coconut oil or peanut oil can be employed for the edible oil solutions. The oil solutions are suitable for intra-articular, intramuscular and subcutaneous injection. The preparation of all these solutions under sterile conditions is readily accomplished by standard pharmaceutical techniques well-known to those skilled in the art.

It is also possible to administer the compounds of the present invention topically when treating inflammatory conditions of the skin and this may preferably be done by way of creams, jellies, gels, pastes, ointments and the like, in accordance with standard pharmaceutical practice.

### EXAMPLES AND PREPARATIONS

The present invention is illustrated by the following examples and preparation. However, it should be understood that the invention is not limited to the specific details of these examples and preparations. Melting points were taken with a Buchi micro melting point apparatus and is not corrected. Infrared Ray absorption spectra (IR) were measured by a Shimadzu infrared spectrometer (IR-470). ¹H and ¹³C nuclear magnetic resonance spectra (NMR) were measured in CDCl₃ by a JEOL NMR spectrometer (JNM-GX270, 270 MHz) unless otherwise indicated and peak positions are expressed in parts per million (ppm) downfield from tetramethylsilane. The peak shapes are denoted as follows: s, singlet; d, doublet; t, triplet; m, multiplet; br, broad.

Analytical data of compounds, which can be prepared according to General Procedures A and B or were prepared in Examples hereinafter disclosed, can be taken by utilizing Waters LC-MS system (LC as 2690, ZMD as MS).

Analytical condition for LC-MS: Column YMC CombiScreen basic 4.6 mm×50 mm, Flow rate 1 mL/min.; Mobile phase 20% MeOH/ 80% 0.1% HCO₂H in H₂O programmed over 5 min to 90% MeOH/10% 0.1% HCO₂H in H₂O Hold for 5 min.; Wave length 220-400 nm. MS detector Apcl Cone 30 Volts.

### Preparation 1

### 2,3-Dihydro-1′-[2-(ethoxycarbonyl)ethyl]spiro[1H-indene-1,4′-piperidine]

A mixture of 2,3-dihydrospiro[1H-indene-1,4′-piperidine] hydrochloride (1.00 g, 4.47 mmol, this was prepared according to known procedure: M. S. Chambers et al, J. Med. Chem. 1992, 35, 2033), ethyl 3-bromopropionate (1.62 g, 8.94 mmol) and N,N-diisopropylethylamine (1.73 g, 13.4 mmol) in EtOH (20 ml) was stirred at 65 ° C. for 18 h. Then the reaction mixture was concentrated, basified with NaHCO₃ solution, and extracted with CH₂Cl₂. The extracts combined were dried (MgSO₄), filtered, and concentrated. The residue was purified by silica gel column chromatography (CH₂Cl₂/MeOH: 40/1 as eluent) to give 1.28 g (99%) of title compound as colorless oil.

¹H NMR (300 MHz, CDCl₃) δ 7.22−7.12 (4H, m), 4.46 (2H, q, J=7.2 Hz), 2.95−2.82(6H, m), 2.80−2.73 (2H, m), 2.60−2.52(2H, m), 2.28−2.18 (2H, m), 2.03−1.87 (4H, m), 1.60−1.50 (2H, m), 1.28 (3H, t, J=7.2 Hz).

MS(EI direct) m/z: 287(M)⁺.

### Preparation 2

### 2,3-Dihydro-1′-[2-(carboxy)ethyl]spiro[1H-indene-1,4′-piperidine] hydrochloride

A mixture of 2,3-dihydro-1′-[2-(ethoxycarbonyl)ethyl]spiro[1H-indene-1,4′-piperidine] (1.28 g, 4.45 mmol), 2N HCl (10 ml) and AcOH (10 ml) was stirred at 100 ° C. for 20 h. After cooling down to 0° C., the resulting white solid appeared was collected by filtration, washed with AcOEt, and dried to afford 1.13 g (86%) of title compound as a white solid.

¹H NMR (300 MHz, DMSO-d6) δ 10.20 (1H, br.s), 7.25−7.10 (4H, m), 3.50−3.00 (6H, m), 2.89−2.82 (4H, m), 2.23−2.08 (2H, m), 2.04 (2H, t, J=7.2Hz), 1.70−1.60 (2H, m).

MS(ESI positive) m/z: 260(M+H)⁺.

### Preparation 3

### 2,3-Dihydro-1′-[2-(chloroformyl)ethyl]spiro[1H-indene-1,4′-piperidine] hydrochloride

To a stirred suspension of 2,3-dihydro-1′-[2-(carboxy)ethyl]spiro[1H-indene-1,4′-piperidine] hydrochloride (0.80 g, 2.70 mmol) in thionyl chloride (6 ml) was added DMF (0.2 ml) at room temperature. After 1 h stirring, the reaction mixture was diluted with mixed solvents (CH₂Cl₂/hexane: 1/1). The resulting solid appeared was collected by filtration and dried to give 0.77 g (91%) of title compound as white solid.

¹H NMR (300 MHz, DMSO-d6) δ 10.81 (1H, br.s), 7.25−7.09 (4H, m), 3.52−3.42 (2^{H, m),} 3.36−3.27 (2H, m), 3.17−3.01 (2H, m), 2.94−2.86 (4H, m), 2.31−2.18 (2H, m), 2.06(2H, t, J=7.2 Hz), 1.69−1.59 (2H, m).

MS(EI direct) m/z: 277(M)⁺.

### Example 1

### 2,3-Dihydro-1′-[3-(2-methoxycarbonylindolin-1 -yl)-3-oxopropyl]spiro[1H-indene-1,4′-piperidine] hydrochloride

To a stirred solution of methyl indoline-2-carboxylate (152 mg, 0.86 mmol) and triethylamine (0.36 ml, 2.58 mmol) in CH₂Cl₂ (5 ml) was added 2,3-dihydro-1′-[2-(chloroformyl)ethyl]spiro[1H-indene-1,4′-piperidine] hydrochloride (270 mg, 0.86 mmol) at room temperature and the resulting reaction mixture was stirred for 5 h. The reaction mixture was poured into a saturated aqueous NaHCO₃ solution and extracted with CH₂Cl₂. The extracts combined were washed with brine, dried (MgSO₄), filtered, and concentrated. The residue was purified by silica gel column chromatography (CH₂Cl₂/MeOH: 30/1 as an eluent) to give 160 mg (44%) of colorless amorphous solid.

¹H NMR (270 MHz, CDCl₃) δ 6 8.28−8.19 (0.5H, m), 7.26−7.10 (6.5H, m), 7.07−7.00 (1H, m), 5.25−5.00(1H, m), 3.77 (3H, br.s), 3.70−3.40 (1H, m), 3.35−2.80 (8H, m) 2.75−2.50 (1H, m), 2.37−2.20 (2H, m), 2.07−1.40 (4H, m), 1.62−1.50 (2H, m).

33 mg of this solid was dissolved in HCl solution in MeOH (1 ml), concentrated, solidified with CH₂Cl₂/hexane, washed with ether, and collected by filtration to give 29 mg of title compound as white amorphous solid.

¹H NMR (270 MHz, CDCl₃) δ 6 12.40 (1H, br.s), 8.18 (0.75H, d, J=8.2 Hz), 7.43−7.30 (1.25H, m), 7.26−7.15 (5H, m), 7.07 (1H, t, J=7.2Hz), 5.25−5.10 (1H, m), 3.85(2.25H, s), 3.74 (0.75H, s), 3.72−3.32 (6H, m), 3.20−2.60 (6H, m), 2.07 (2H, t, J=7.1 Hz), 1.80−1.50(4H, m).

MS (ESI positive) m/z: 419 (M+H)⁺.

IR(KBr): 3310, 2934, 2561, 1744, 1655, 1481, 1418, 1207, 758 cm⁻¹

Anal. Calcd for C₂₆H₃₀N₂O₃—HCl-0.8H₂O: C, 66.53; H, 7.00; N, 5.97. Found: C, 66.55; H, 7.00; N, 5.97.

### Preparation 4

### 2,3-Dihydro-1′-[2-(2-hydroxyethoxycarbonyl)ethyl]spiro[1H-indene-1,4′-piperidine]

A mixture of 2,3-dihydrospiro[1H-indene-1,4′-piperidine] hydrochloride (0.31 g, 1.39 mmol, this was prepared according to known procedure: M. S. Chambers et al, J. Med. Chem. 1992, 35, 2033), ethyl 3-bromopropionate (0.50 g, 2.77 mmol) and N,N-diisopropylethylamine (0.54 g, 4.17 mmol) in ethylene glycol (10 ml) was stirred at 80 ° C. for 16 h. Then the reaction mixture was poured into a saturated aqueous NaHCO3 solution, and extracted with AcOEt. The extracts combined were dried (MgSO₄), filtered, and concentrated. The residue was purified by silica gel column chromatography (CH₂Cl₂/MeOH: 20/1 as an eluent) to give 0.37 g (88%) of title compound as colorless oil.

¹H NMR (300 MHz, CDCl₃) δ 7.25−7.15 (4H, m), 4.37−4.33 (2H, m), 3.84−3.78 (2H, m), 3.01−2.94 (2H, m), 2.94 (2H, t, J=8.1 Hz), 2.78−2.72 (2H, m), 2.64−2.58 (2H, m), 2.14−2.05(2H, m), 2.04−1.91 (4H, m, including 2H, t, J=8.1 Hz at 2.00 ppm), 1.60−1.50(8H, m). MS(EI direct) m/z: 303(M)⁺.

### Preparation 5

### 2,3-Dihydro-1′-[2-(carboxy)ethyl]spiro[1H-indene-1,4′-piperidine]

A mixture of 2,3-dihydro-1′-[2-(2-hydroxyethoxycarbonyl)ethyl]spiro[1H-indene-1,4′-piperidine] (0.37 g, 1.22 mmol), 2N NaOH (4 ml) and EtOH (10 ml) was refluxed with stirring for 16 h. After cooling down to 0° C., the resulting mixture was neutralized with a 2N HCl solution and extracted with CH₂Cl₂ and AcOEt. The extracts combined were dried (MgSO₄), filtered, and concentrated to give 120 mg (38%) of title compound as an yellow solid.

¹H NMR (270 MHz, CDCl₃) δ 7.26−7.20 (4H, m), 3.52−3.43 (2H, m), 3.25−3.15 (2H, m), 2.96 (2H, t, J=8.1Hz), 2.91−2.81 (2H, m), 2.70−2.63 (2H, m), 2.33−2.19 (2H, m), 2.08 (2H, t, J=8.1 Hz), 1.81−1.70 (2H, m).

### Example 2

### 2,3-Dihydro-1′-[3-(indolin-1-yl)-3-oxopropyl]spiro[1H-indene-1,4′-piperidine] hydrochloride

A mixture of 2,3-dihydro-1′-[2-(carboxy)ethyl]spiro[1H-indene-1,4′-piperidine] (14 mg, 0.054 mmol), indoline (12 μl, 0.108 mmol), WSC (21 mg, 0.108 mmol), HOBt (15 mg, 0.108 mmol), and triethylamine (23 μl, 0.162 mmol) in CH₂Cl₂ (3 ml) was stirred at room temperature overnight. A saturated aqueous NaHCO₃ solution was added to the reaction mixture and aqueous layer was removed by decantation. The separated organic layer was dried (MgSO₄), filtered, and concentrated. The resulting residue was purified by preparative TLC (1 mm thick silica gel plate: CH₂Cl₂/MeOH: 10/1) to afford 12 mg (62%) of colorless oil.

¹H NMR (270 MHz, CDCl₃) δ 8.24 (1H, d, J=8.11Hz), 7.24−7.12 (6H, m), 7.05−6.98(1H, m), 4.10 (2H, t, J=8.4Hz), 3.21 (2H, t, J=8.4Hz), 3.00−2.86 (8H, m), 2.76−2.68(2H, m) 2.36−2.24 (2H, m), 2.03 (2H, t, J=7.2Hz), 2.03−1.90 (2H, m), 1.63−1.53 (2H, m).

This was converted to HCl salt similar to that described in Example 1 to afford 12 mg of title compound as white solid.

MS (ESI positive) m/z: 361 (M+H)⁺.

### Example 3

### 2,3-Dihydro-1′-[3-(benzimidazol-2-one-1-yl)propyl]spiro[1H-indene-1,4′-piperidine]

In a one-dram vial were mixed a solution of 1-(3-bromopropyl)benzimidazol-2-one (38 mg, 0.15 mmol, this was reported in EP181793) in ethylenelycol (1 ml) and a solution of 2,3-dihydrospiro[1H-indene-1,4′-piperidine] hydrochloride (11 mg, 0.05 mmol) and N,N-diisopropylethylamine (17 μl1, 0.1 mmol) in ethyleneglycol (1 ml), and the mixture was agitated by shaking at 100° C. After 24 h, the reaction mixture was loaded onto a BondElute® SCX cartridge (500 mg /3 ml) which was preconditioned with MeOH (1 ml). The solid-phase matrix was washed with MeOH (5 ml) and then eluted with 2M ammonia/MeOH solution (2 ml). The eluate was concentrated under reduced pressure to give an oil, to which were added CH₂Cl₂ (1 ml) and PS-NCO (1.3 mmol/g; 75 mg, 0.1 mmol). The resulting suspension was shaken at room temperature for 2 h. Insoluble polymers were removed by filtration, and the filtrate was concentrated to dryness by vacuum centrifuge to give an amorphous solid, which was purified with reverse-phase preparatory HPLC (0.1% HCO2H-MeOH) to give the title compound as a formic acid salt (6.2 mg; 27% yield).

ESI-MS (LC/MS): Calcd. for C₂₃H₂₇N₃O:[M+H]⁺=362.22. Found: 362.58

HPLC purity: 97.8% (UV 210-400 nm); retention time: 3.58 min

### Preparation 6

### 2,3-Dihydro-1′-(3-hydroxypropyl)spiro[1H-indene-1,4′-piperidine]

A mixture of 2,3-dihydrospiro[1H-indene-1,4′-piperidine] hydrochloride (0.5 g, 2.23 mmol, this was prepared according to known procedure: M. S. Chambers et al, J. Med. Chem. 1992, 35, 2033), 3-bromopropanol (0.3 ml, 3.35 mmol), K₂CO₃ (924.6 mg, 6.69 mmol), and Kl (185.9 mg, 1.12 mmol) in MeCN (30 ml) was refluxed with stirring for 18 h. After cooling down to room temperatute, water (30 ml) was added to the reaction mixture and extracted with CH₂Cl₂ (20 ml×3). The extracts combined were dried (Na₂SO₄), filtered, and concentrated to give 574.7 mg of crude product. This was purified by silica gel column chromatography (CH₂Cl₂/MeOH: 15/1 as an eluent) to afford 288.7 mg (53%) of title compound as pale yellow white solid.

¹H NMR (270 MHz, CDCl₃) δ 7.26−7.12 (4H, m), 3.86 (2H, t, J=5.3Hz), 3.34−3.24(2H, m), 2.95−2.88 (4H, m), 2.56−2.42 (2H, m), 2.26−2.10 (2H, m), 2.03 (2H, t, J=7.3 Hz), 1.96−1.85(2H, m), 1.71−1.60 (2H, m).

MS(EI direct) m/z: 245(M)⁺.

### Preparation 7

### 2,3-Dihydro-1′-(3-mesyloxypropyl)spiro[1H-indene-1,4′-piperidine]

To a stirred solution of 2,3-dihydro-1′-(3-hydroxypropyl)spiro[1H-indene-1,4′-piperidine] (288.7 mg, 1.18 mmol) in CH₂Cl₂ (10 ml) was added triethylamine (0.3 ml, 2.12 mmol) followed by dropwise addition of mesyl chloride (0.11 ml, 1.42 mmol) at 0 ° C. After 1 h stirring at 0 ° C., the reaction mixture was poured into a saturated aqueous NaHCO₃ solution and extracted with CH₂Cl₂ (30 ml×3). The extracts combined were washed with brine, dried (Na₂SO₄), filtered, and concentrated to give 330.4 mg of title compound as yellow oil, which was used for the next reaction without purification.

¹H NMR (270 MHz, CDCl₃) δ 7.26−7.11 (4H, m), 4.34 (2H, t, J=6.4 Hz), 3.03 (3H, s), 2.96−2.80 (4H, m), 2.51 (2H, t, J=7.2Hz), 2.24−2.12 (2H, m), 2.05−1.84 (6H, m), 1.62−1.50(2H, m).

MS(EI direct) m/z: 323(M)⁺.

### Example 4

### 2,3-Dihydro-1′-[3-(benzothiazol-2-one-1-yl)propyl]spiro[1H-indene-1,4′-piperidine]

To a stirred solution of NaH (13.6 mg, 0.34 mmol, 60% oil dispersion in mineral oil, which was removed by washing with n-hexane (2 ml×2) before use) and benzothiazol-2-one (46.9 mg, 0.31 mmol) in DMF (1 ml) was added a solution of 2,3-dihydro-1′-(3-mesyloxypropyl)spiro[1H-indene-1,4′-piperidine] (50 mg, 0.155 mmol) in DMF (1.5 ml) at 0 ° C. The reaction mixture was heated to 100 ° C. with stirring for 21 h. The reaction mixture was cooled to 0 ° C. and NaHCO₃ solution was added to the reaction mixture, then extracted with CH₂Cl₂ (15 ml×3). The extracts combined were washed with brine, dried (Na₂SO₄), and filtered. The filtrate was evaporated in vacuo to afford 87 mg of crude product, which was purified by preparative TLC (1 mm thick silica gel plate: CH₂Cl₂/MeOH:20/1, 2 times developed) to give the product. It was purified again by preparative TLC (1 mm thick silica gel plate: n-hexane/AcOEt:2/1, 2 times developed) to give 36.4 mg (62%) of the title compound as pale yellow oil.

¹H NMR (270 MHz, CDCl₃) δ 7.45−7.41 (1H, m), 7.35−7.28 (1H, m), 7.24−7.12 (6H, m), 4.05 (2H, t, J=6.9Hz), 2.92−2.80 (4H, m), 2.46 (2H, t, J=6.9 Hz), 2.19−2.08(2H, m), 2.04-1.83 (6H, m), 1.58−1.48 (2H, m).

MS (ESI positive) m/z: 379 (M+H)⁺.

This was converted to HCl salt similar to that described in Example 1 to give 24.7 mg of HCl salt as white solid.

IR(KBr): 3416, 2939, 2500, 1678, 1474, 748 cm⁻¹

Anal. Calcd for C₂₃H₂₆N₂OS—HCl-0.4H₂O: C, 65.43; H, 6.64; N, 6.63. Found: C, 65.66; H, 6.81; N, 6.36.

### Preparation 8

### 2,3-Dihydro-1′-[3-(2-carboxyindol in-1-yl)-3-oxopropyl]spiro[1H-indene-1,4′-piperidine]

A mixture of 2,3-dihydro-1′-[3-(2-methoxycarbonylindolin-1-yl)-3-oxopropyl]spiro[1H-indene-1,4′-piperidine] (42 mg, 0.092 mmol, this was prepared in Example 1) and 2N HCl (1 ml) in acetic acid (3 ml) was heated at 90 ° C. with stirring for 16 h. The reaction mixture was concentrated to give solid which was triturated in AcOEt. The solid was collected by filtration to afford 30 mg as a pale red solid. This showed no methyl singlet peak of methyl ester in starting material in 1H NMR spectroscopy. This was used for the next reaction without purification.

### Example 5

### 2,3-Dihydro-1′-{3-[2-(N-methylaminocarbonyl)indolin-1 -yl]-3-oxopropyl}spiro[1H-indene-1,4′-piperidine]

A mixture of 2,3-dihydro-1′-[3-(2-carboxyindolin-1 -yl)-3-oxopropyl]spiro[1H-indene-1,4′-piperidine] (30 mg, 0.068 mmol), methylamine hydrochloride (10 mg, 0.136 mmol), WSC (26 mg, 0.136 mmol), HOBt (19 mg, 0.136 mmol), and triethylamine (47 μl, 0.34 mmol) in CH₂Cl₂ (4 ml) was stirred at room temperature for 16 h. The reaction mixture was poured into saturated aqueous NaHCO₃ solution, extracted with CH₂Cl₂, dried (MgSO₄), filtered, and concentrated. The residue was purified by preparative TLC (1 mm thick silica gel plate, CH₂Cl₂/MeOH: 10/1) to afford 6 mg (21%) of title compound as white solid.

¹H NMR (270 MHz, CDCl₃) δ 8.20 (1H, br.s), 7.26−7.00 (7H, m), 6.40 (1H, br.s), 5.30−4.90 (1H, m), 3.75−3.20 (2H, m), 3.10−2.90 (4H, m), 2.90 (2H, t, J=7.4Hz), 2.79(3H, d, J=4.8 Hz), 2.45−2.25 (4H, m), 2.02 (2H, t, J=7.4 Hz), 2.09−1.90 (2H, m), 1.63−1.53(2H, m).

MS (ESI positive) m/z: 418 (M+H)⁺.

This was converted to HCl salt similar to that described in Example 1 to give 6 mg of HCl salt as a pale gray solid.

MS (ESI positive) m/z: 418 (M+H)⁺.

### Example 6

### 2,3-Dihydro-1′-[2-(1,1-dioxido-3-oxo-1,2-benzisotiazol-2(3H)-yl)ethyl]spiro[1H-indene-1,4′-piperidine]

A mixture of 2,3-dihydrospiro[1H-indene-1,4′-piperidine] hydrochloride (80 mg, 0.357 mmol), N-2-(mesyloxy)ethylsaccharin (130.7 mg, 0.428 mmol), K₂CO₃ (148 mg, 1.07 mmol) and Kl (29.7 mg, 0.179 mmol) in MeCN (6 ml) was refluxed with stirring for 18 h. After cooling down to room temperature, the reaction mixture was poured into aqueous NaHCO₃ solution and extracted with CH₂Cl₂ (20 ml×3). The extracts combined were washed with brine, dried (Na₂SO₄), filtered, and concentrated to give 191.7 mg of crude product, which was purified by preparative TLC (1 mm thick silica gel plate, CH₂Cl₂/MeOH: 25/1). Then extracted product was purified again by preparative TLC (n-hexane/AcOEt:1/1, 2 times developed) to give 31.6 mg (22%) of title compound as pale yellow oil.

¹H NMR (270 MHz, CDCl₃) δ 8.10−8.05 (1H, m), 7.96−7.80 (3H, m), 7.24−7.12 (4H, m), 3.96 (2H, dd, J=7.2, 7.6 Hz), 3.04−2.95 (2H, m), 2.89 (2H, t, J=7.4 Hz), 2.85(2H, t, J=7.6 Hz), 2.41−2.28 (2H, m), 2.06−1.88 (4H, m), 1.96−1.88 (2H, m).

MS (ESI positive) m/z: 397 (M+H)⁺.

IR(KBr): 2924, 1734, 1327, 1180, 752 cm⁻¹

Anal. Calcd for C₂₂H₂₄N₂O₃S-0.2H₂O: C, 66.04; H, 6.15; N, 7.00. Found: C, 66.06; H, 6.27; N, 6.73.

### Example 7

### 2,3-Dihydro-1′-[3-(2-oxo-3,4-dihydro-1(2H)-quinolinyl)propyl]spiro[1H-indene-1,4′-piperidine]

This was prepared according to the procedure described in Example 4 using 3,4-dihydro-2(1H)-quinolinone instead of benzothiazol-2-one. Yield was 38.1 mg (66%). Product was pale yellow oil.

¹H NMR (270 MHz, CDCl₃) δ 8 7.28−7.10 (7H, m), 6.99 (1H, ddd, J=1.2, 7.2, 7.4 Hz), 4.02 (2H, dd, J=7.3, 7.6 Hz), 2.95−2.84 (6H, m), 2.68−2.61 (2H, m), 2.52−2.45(2H, m), 2.226-2.12 (2H, m), 2.03−1.84 (6H, m), 1.60−1.50 (2H, m).

To a stirred solution of this oil (36.3 mg, 0.097 mmol) in MeOH (1.5 ml) was added citric acid (18.6 mg, 0.097 mmol) at room temperature. After 2 h stirring, the solvent was evaporated to give 45 mg of citric acid salt as white amorphous solid.

MS (ESI positive) m/z: 375 (M+H)⁺.

IR(KBr): 3402, 2945, 2600, 1728, 1657, 1601, 1387, 1190, 758 cm⁻¹

Anal. Calcd for C₂₅H₃₀N₂O—C₆H₈O₇—H₂O: C, 63.68; H, 6.90; N, 4.79. Found: C, 63.90; H, 6.86; N, 4.63.

### Example 8

### 2,3-Dihydro-1′-[3-(3-methyl-2-oxo-3,4-dihydro-1(2H)-quinazolinyl)propyl]spiro[1H-indene-1,4′-piperidine]

This was prepared according to the procedure described in Example 4 using 3,4-dihydro-3-methyl-2(1H)-quinazolinone instead of benzothiazol-2-one. Yield was 28 mg (46%). Product was pale yellow oil.

¹H NMR (270 MHz, CDCl₃) δ 7.28−7.10 (5H, m), 7.08−6.91 (3H, m), 4.37 (2H, s), 3.94(2H, dd, J=7.4, 7.6Hz), 3.02 (3H, s), 3.01−2.86 (4H, m), 2.58−2.50 (2H, m), 2.29−2.16 (2H, m), 2.06−1.88 (6H, m), 1.62−1.50 (2H, m).

To a stirred solution of this oil (28 mg, 0.072 mmol) in MeOH (1.5 ml) was added citric acid (13.8 mg, 0.072 mmol) at room temperature. After 1 h stirring, the solvent was evaporated to give 36.8 mg of citric acid salt as white amorphous solid.

MS (ESI positive) m/z: 390 (M+H)⁺.

IR(KBr): 3416, 2939, 2600, 1728, 1657, 1641, 1605, 1489, 1213, 758 cm⁻¹

Anal. Calcd for C₂₅H₃₁N₃O—C₆H₈O₇—H₂O: C, 62.09; H, 6.89; N, 7.01. Found: C, 62.26H, 6.88; N, 6.75.

### Example 9

### 2,3-Dihydro-1′-[3-(2-oxo-1,3-benzoxazol-3(2H)-yl)propyl]spiro[1H-indene-1,4′-piperidine]

This was prepared according to the procedure described in Example 4 using benzoxazol-2-one instead of benzothiazol-2-one. Yield was 29.4 mg (52%). Product was reddish brown oil.

¹H NMR (300 MHz, CDCl₃) δ 7.26−7.06 (8H, m), 3.94 (2H, t, J=6.8 Hz), 2.88 (2H, t, J=7.3 Hz), 2.45 (2H, t, J=6.8 Hz), 2.16−2.06 (2H, m), 2.05−1.94 (4H, m), 1.90−1.78(2H, m), 1.55−1.47 (2H, m).

To a stirred solution of this oil (29.4 mg, 0.081 mmol) in MeOH (1.5 ml) was added citric acid (15.6 mg, 0.081 mmol) at room temperature. After 1 h stirring, the solvent was evaporated to give 32.5 mg of citric acid salt as red amorphous solid.

MS (ESI positive) m/z: 363 (M+H)⁺.

IR(KBr): 3437, 2939, 2544, 1771, 1732, 1589, 1487, 1371, 1254, 756 cm⁻¹

Anal. Calcd for C₂₃H₂₆N₂O₂—C₆H₈O₇-0.5H₂O: C, 61.80; H, 6.26; N, 4.97. Found: C, 61.41; H, 6.24; N, 4.88.

### Example 10

### 2,3-Dihydro-1′-[3-(2-carboxyindolin-1-yl)-3-oxopropyl]spiro [1H-indene-1,4′-piperidine]

To a stirred solution of 2,3-dihydro-1′-[3-(2-methoxycarbonylindolin-1-yl)-3-oxopropyl]spiro[1H-indene-1,4′-piperidine] (125 mg, 0.3 mmol, this was prepared in Example 1) in THF (3 ml) and MeOH (1 ml) was added 2N NaOH (0.6 ml, 1.2 mmol) at room temperature. After 16 h stirring at room temperature, the reaction mixture was neutralized with 2N HCl (0.6 ml) and 4 drops of saturated aqueous NaHCO₃ solution, diluted with water (5 ml), and extracted with CH₂Cl₂. The extracts combined were dried (MgSO₄), filtered, and concentrated to give 105 mg (87%) of title product as white solid.

¹H NMR (270 MHz, DMSO-d6) δ 8.09 (1H, d, J=8.4 Hz), 7.30-6.80 (8H, m), 5.35-5.15 (1H, m), 3.70-2.75 (12H, m), 2.10-1.95 (4H, m), 1.70-1.55 (2H, m).

MS (ESI positive) m/z: 405 (M+H)⁺.

### Example 11

### 2,3-Dihydro-1′-[3-(2-N,N-dimethylaminocarbonylindolin-1-yl)-3-oxopropyl]spiro[1H-indene-1,4′-piperidine] hydrochloride

A mixture of 2,3-dihydro-1′-[3-(2-carboxyindolin-1-yl)-3-oxopropyl]spiro [1H-indene-1,4′-piperridine] (23 mg, 0.057 mmol, this was prepared in Example 10), dimethylamine hydrochloride (14 mg, 0.17 mmol), WSC (22 mg, 0.114 mmol), HOBt (16 mg, 0.114 mmol), and triethylamine (40 μl, 0.29 mmol) in CH₂Cl₂ (3 ml) was stirred at room temperature for 20 h. The reaction mixture was diluted with saturated aqueous NaHCO3 solution and extracted with CH₂Cl₂. The extracts combined were dried (MgSO₄), filtered, and concentrated. The residue was purified by preparative TLC (1 mm thick plate, CH₂Cl₂/MeOH: 10/1) to give 20 mg (81%) of free form of title product as colorless oil.

¹H NMR (270 MHz, CDCl₃) δ 8.29 (0.5H, d, J=7.9 Hz), 7.65-6.95 (7.5H, m), 5.50-5.40 (0.5H, m), 5.35-5.25 (0.5H, m), 3.77-3.60 (0.5H, m), 3.53-3.35 (0.5H, m), 3.22-2.20 (17H, m, including 1.5H, s at 3.19 ppm, 1.5H, s at 3.16 ppm, 1.5H, s at 3.01 ppm, 1.5H, s at 2.98 ppm, 2H, t, J=7.4 Hz at 2.90 ppm), 2.15-1.90 (4H, m, including 2H, t, J=7.4 Hz at 2.02 ppm, 1.50 (2H, m).

This was converted to HCl salt similar to that described in Example 1 to give 15 mg of HCl salt as a white solid.

¹H NMR (270 MHz, CDCl₃) δ 12.13 (1H, br.s), 8.25 (1H, d, J=8.2 Hz), 7.40-7.00 (7H, m), 5.65-5.50 (1H, m), 3.85-2.50 (18H, m including 3H, s at 3.28 ppm, 3H, s at 3.05 ppm, and 2H, t, J=7.4 Hz at 2.95 ppm), 2.04 (2H, t, J=7.4 Hz), 1.80-1.50 (4H, m).

MS (ESI positive) m/z: 432 (M+H)⁺.

IR(KBr): 3446, 2936, 2561, 1653, 1483, 1458, 1398, 1271, 758 cm⁻¹

Anal. Calcd for C₂₇H₃₃N₃O₂—HCl—H₂O: C, 66.72; H, 7.47; N, 8.65. Found: C, 66.48; H, 7.48; N, 8.56.

### Example 12

### 2,3-Dihydro-1′-[3-(2-morpholinocarbonylindolin-1-yl)-3-oxopropyl]spiro [1H-indene-1,4′-piperidine] hydrochloride

This was prepared according to the procedure described in Example 11 using morpholine instead of dimethylamine hydrochloride. 23 mg (86%) of free form of title compound was obtained as colorless oil.

¹H NMR (270 MHz, CDCl₃) δ 8.35-8.23 (0.4H, m), 7.33-7.05 (6.6H, m), 7.01 (1H, br.dd, J=7.4, 8.4 Hz), 5.50-5.40 (0.6H, m), 5.37-5.25 (0.4H, m), 3.90-3.35 (9H, m), 3.13-2.20 (11H, m, including 2H, t, J=7.5 Hz at 2.90 ppm), 2.10-1.90 (4H, m, including 2H, t, J=7.4 Hz at 2.02 ppm), 1.65-1.50 (2H, m).

This was converted to HCl salt similar to that described in Example 1 to give 18 mg of HCl salt as a white solid.

¹H NMR (270 MHz, CDCl₃) δ 8.25 (1H, d, J=7.9 Hz), 7.40-7.00 (8H, m), 5.80-5.70 (1H, m), 4.08-3.35 (13H, m), 3.13-2.50 (7H, m, including 2H, t, J=7.4 Hz at 2.95 ppm), 2.04 (2H, t, J=7.6 Hz), 1.80-1.50 (4H, m).

MS (ESI positive) m/z: 474 (M+H)⁺.

IR(KBr): 2928, 2550, 1655, 1119, 752 cm⁻¹

Anal. Calcd for C₂₉H₃₅N₃O₃—HCl-0.7H₂O: C, 66.64; H, 7.21; N, 8.04. Found: C, 66.85; H, 7.32; N, 7.89.

### Example 13

### 2,3-Dihydro-1′-[3-(2-carboxyindolin-1-yl)-3-oxopropyl]spiro [1H-indene-1,4′-piperidine] hydrochloride

To a stirred suspension of 2,3-dihydro-1′-[3-(2-carboxyindolin-1-yl)-3-oxopropyl]spiro[1H-indene-1,4′-piperidine] (20 mg, 0.049 mmol, this was prepared in Example 10) in MeCN (4 ml) was added 1,1′-carbonyldiimidazole (9 mg, 0.054 mmol) at room temperature and resulting mixture was refluxed for 0.5 h. Triethylamine (10 μl) was added to the reaction mixture and reflux was continued for 2 h. To a reaction mixture was added 25% NH₄OH (2 ml) and reflux was continued for 2 h. Then the reaction mixture was concentrated, diluted with saturated aqueous NaHCO₃ solution, and extracted with CH₂Cl₂. The extracts combined were dried (MgSO₄), filtered, and concentrated. The residue was purified by preparative TLC (1 mm thick plate, CH₂Cl₂/MeOH: 10/1) to afford 9 mg (45%) of free form of title compound as colorless amorphous solid. This compound showed broadened spectra in proton NMR.

This was converted to HCl salt similar to that described in Example 1 to give 8 mg of HCl salt as a white solid.

¹H NMR (270 MHz, CDCl₃ +CD₃OD) δ 8.17 (1H, d, J=7.6 Hz), 7.38-7.03 (8H, m), 5.35-5.10 (1H, m), 3.85-3.20 (10H, m), 3.15-2.35 (6H, m, including 2H, t, J=7.3 Hz at 3.00 ppm), 2.10 (2H, t, J=7.3 Hz), 1.83-1.70 (2H, m).

MS (ESI positive) m/z: 404 (M+H)⁺.

### Example 14

### 2,3-Dihydro-1′-[3-(2-(S)-methoxycarbonylindolin-1-yl)-3-oxopropyl]spiro [1H-indene-1,4′-piperidine] hydrochloride

To a stirred suspension of (2S)-methyl indoline-2-carboxylate hydrochloride (520 mg, 2.43 mmol) in CH2Cl2 (10 ml) was added triethylamine (1.13 ml, 8.1 mmol) at 0° C. After 10 minutes stirring, 2,3-dihydro-1′-[2-(chloroformyl)ethyl]spiro[1H-indene-1,4′-piperidine] hydrochloride (510 mg, 1.62 mmol) was added to the reaction mixture at 0° C. and the resulting reaction mixture was stirred at 0° C. for 4 h. The reaction mixture was quenched with a saturated aqueous NaHCO₃ solution and extracted with CH₂Cl₂. The extracts combined were washed with brine, dried (MgSO4), filtered, and concentrated. The residue was purified by silica gel column chromatography (CH₂Cl₂/MeOH: 20/1 as an eluent) to give 345 mg (49%) of colorless amorphous solid.

¹H NMR (270 MHz, CDCl₃) δ 8.30-8.15 (0.5H, m), 7.35-7.07 (6.5H, m), 7.05-6.95 (1H, m), 5.25-4.98(1H, m), 3.74 (3H, br.s), 3.70-3.35 (1H, m), 3.35-2.45 (9H, m), 2.35-2.15 (2H, m), 2.05-1.85 (4H, m), 1.65-1.48 (2H, m).

24 mg of this solid was dissolved in HCl solution in MeOH (0.5 ml), concentrated, solidified with ether, and collected by filtration to give 22 mg of title compound as white amorphous solid.

MS (ESI positive) m/z: 419 (M+H)⁺.

IR(KBr): 3420, 2951, 2563, 1744, 1661, 1481, 1418, 1207, 758 cm⁻¹

Anal. Calcd for C₂₆H₃₀N₂O₃—HCl-0.6H₂O: C, 67.04; H, 6.97; N, 6.01. Found: C, 67.07; H, 7.10; N, 5.78.

### Example 15

### 2,3-Dihydro-1′-{3-[2-(1-ethylprrolydin-3-yl) aminocarbonylindolin-1yl]-3-oxopropyl}spiro[1H-indene-1,4′-piperidine] dihydrochloride

A mixture of 2,3-dihydro-1′-[3-(2-carboxyindolin-1-yl)-3-oxopropyl]spiro [1H-indene-1,4′-piperidine] (35 mg, 0.087 mmol, this was prepared in Example 10), 3-amino-1-benzylpyrrolidine (31 mg, 0.17 mmol), WSC (33 mg, 0.17 mmol), HOBt (23 mg, 0.17 mmol), and triethylamine (36 μl, 0.26 mmol) in CH₂Cl₂ (4 ml) was stirred at room temperature for 18 h. The reaction mixture was diluted with saturated aqueous NaHCO₃ solution and extracted with CH₂Cl₂. The extracts combined were dried (MgSO₄), filtered, and concentrated. The residue was purified by preparative TLC (1 mm thick plate, CH₂Cl₂/MeOH: 7/1) to give 28 mg (57%) of amide product as colorless oil.

MS (ESI positive) m/z: 563 (M+H)⁺.

A suspension mixture of this oil (28 mg, 0.05 mmol), 10% palladium on activated carbon (10 mg) and EtOH (6 ml) was stirred under hydrogen atmosphere at room temperature for 24 h. Then 5 mg of 10% palladium on activated carbon was added to the reaction mixture and continued the hydrogenation for 24 h. After the removal of the catalyst by filtration, the filtrate was concentrated. The resulting crude oil was purified by preparative TLC (1 mm thick plate, CH₂Cl₂/MeOH: 7/1) to give 15 mg (64%) of pale brown oil as free form of title compound. This compound showed broadened spectra in proton NMR. This was converted to HCl salt similar to that described in Example 1 to give 15 mg of HCl salt as a white solid.

MS (ESI positive) m/z: 501 (M+H)⁺.

### Example 16

### 2,3-Dihydro-1′-indolyl-3-oxopropylspiro[1H-indene-1,4′-piperidine] hydrochloride

To a stirred suspension of 2,3-dihydro-1-[2-(chloroformyl)ethyl]spiro[1H-indene-1,4′-piperidine] hydrochloride (100 mg, 0.32 mmol), indole (75 mg, 0.64 mmol), tetrabutylammonium hydrogen sulfate (54 mg, 0.16 mmol) and powdered NaOH (51 mg, 1.28 mmol) in CH₂Cl₂ (4 ml) was added triethylamine (67 μl, 0.48 mmol) at room temperature. After 45 minutes stirring, the reaction mixture was quenched with a saturated aqueous NaHCO₃ solution and extracted with CH₂Cl₂. The extracts combined were washed with brine, dried (MgSO₄), filtered, and concentrated. The residue was purified by preparative TLC (1 mm thick plate, CH₂Cl₂/MeOH: 10/1, then purified again using 0.5 mm thick plate, ethyl acetate) to give 7 mg (6%) of colorless oil.

¹H NMR (270 MHz, CDCl₃) δ 8.47 (1H, d, J=8.2 Hz), 7.57 (1H, d, J=8.2 Hz), 7.51 (1H, d, J=3.8 Hz), 7.40-7.12 (6H, m), 6.66 (1H, d, J=3.8 Hz), 3.20 (2H, t, J=6.9 Hz), 3.06-2.87 (6H, m), 2.40-2.28 (2H, m), 2.07-1.91 (4H, m), 1.64-1.54 (2H, m).

7 mg (0.02 mmol) of this oil and citric acid (3.8 mg, 0.02 mmol) was dissolved in CH₂Cl₂ (1 ml) and MeOH (1 ml) mixture. After 1 h stirring, the mixture solution was concentrated, solidified with ether, and collected by filtration to give 6 mg of title compound as white amorphous solid.

MS (ESI positive) m/z: 359 (M+H)⁺.

### Preparation 9

### 2,3-Dihydro-1′-[3-(2-(S)-carboxyindolin-1-yl)-3-oxopropyl]spiro[1H-indene-1, 4,′-piperidine]

This was prepared according to the procedure described in Example 10 using 2,3-dihydro-1′-[3-(2-(S)-methoxycarbonylindolin-1-yl)-3-oxopropyl]spiro[1H-indene-1,4′-piperidine] instead of 2,3-dihydro-1-[3-(2-methoxycarbonylindolin-1-yl)-3-oxopropyl]spiro [1H-indene-1,4′-piperidine]. 300 mg (100%) of title compound was obtained as white solid.

¹H NMR (270 MHz, CDCl₃) δ 8.22 (1H, d, J=7.9 Hz), 7.24-7.08 (6H, m), 7.04-6.97 (1H, m), 6.94-6.86 (1H, m), 5.06-4.97 (1H, m), 3.70-3.06 (8H, m), 3.00-2.76 (4H, m), 2.33-2.13 (2H, m), 2.06-1.94 (2H, m), 1.68-1.44 (2H, m).

MS (ESI positive) m/z: 405 (M+H)⁺.

### Example 17

### 2,3-Dihydro-1′-{3-[2-(S)-(N,N-dimethylaminoethyl)aminocarbonylindolin-1-yl]-3-oxopropyl}spiro[1H-indene-1,4′-piperidine] dihydrochloride

A mixture of 2,3-dihydro-1′-[3-(2-(S)-carboxyindolin-1-yl)-3-oxopropyl]spiro [1H-indene-1,4′-piperidine] (50 mg, 0.124 mmol, this was prepared in Preparation 9), N,N-dimethylethylenediamine (41 μl, 0.37 mmol), WSC (48 mg, 0.25 mmol), HOBt (34 mg, 0.25 mmol), and triethylamine (86 μl, 0.62 mmol) in CH₂Cl₂ (3 ml) was stirred at room temperature for 18 h. The reaction mixture was diluted with saturated aqueous NaHCO₃ solution and extracted with CH₂Cl₂. The extracts combined were dried (MgSO₄), filtered, and concentrated. The residue was purified by preparative TLC (1 mm thick plate, CH₂Cl₂/MeOH: 5/1) to give 37 mg (63%) of free form of title compound as colorless oil. This compound showed broadened spectra in proton NMR.

This oil was converted to citric acid salt by mixing with 2 equivalent of citric acid in mixed solvent of CH₂Cl₂-MeOH followed by concentration.

MS (ESI positive) m/z: 475 (M+H)⁺.

IR(KBr): 3398, 2941, 2712, 1728, 1655, 1595, 1483, 1418, 1215, 760 cm⁻¹

Anal. Calcd for C₂₉H₃₈N₄O₂-2C₆H₈O₇—H₂O: C, 56.16; H, 6.44; N, 6.39. Found: C, 55.82; H, 6.44; N, 6.22.

### Preparation 10

### 2,3-Dihydro-1′-(3-phthalimidopropyl)spiro[1H-indene-1,4′-piperidine]

This was prepared according to the procedure described in Preparation 6 using N-(3-bromopropyl) phthalimide instead of 3-bromopropanol. 1184 mg (71%) of title compound was obtained as yellow solid.

¹H NMR (270 MHz, CDCl₃) δ 7.91-7.83 (2H, m), 7.77-7.70 (2H, m), 7.20-7.08(3H, m), 6.97-6.88 (1H, m), 3.80 (2H, t, J=6.8 Hz), 2.88-2.78 (4H, m), 2.47 (2H, t, J=6.9 Hz), 2.11-2.00 (2H, m), 1.98-1.88 (4H, m), 1.74-1.60 (2H, m), 1.48-1.38 (2H, m).

MS (ESI, direct) m/z: 374 (M)⁺.

### Preparation 11

### 2,3-Dihydro-1′-[3-(2-nitroanilino)propyl]spiro[1H-indene-1,4′-piperidine]

A mixture of 2,3-dihydro-1′-(3-phthalimidopropyl)spiro[1H-indene-1,4′-piperidine] (1.184 g, 3.16 mmol, this was prepared in preparation 10) and hydrazine hydrate (0.348 g, 6.95 mmol) in MeOH (35 ml) was refluxed with stirring for 2 h. After concentration, the reaction mixture was diluted with aqueous NaHCO3 solution (80 ml) and extracted with CH₂Cl₂ (50 ml×3). The extracts combined were washed with water (50 ml), dried (Na₂SO₄), filtered, and concentrated to give 381.4 mg (crude yield was 49%) of amine derivative as yellow oil.

¹H NMR (270 MHz, CDCl₃) δ 7.23-7.10 (4H, m), 2.93-2.55 (6H, m), 2.50-2.41 (2H, m), 2.20-2.08 (2H, m), 2.05-1.88 (4H, m), 1.75-1.63 (2H, m), 1.60-1.50 (2H, m), 1.40 (2H, br.s).

A mixture of above amine derivative (607 mg, 2.48 mmol), 2-fluoronitrobenzene (0.39 ml, 3.72 mmol), and K₂CO₃ (514 mg, 3.72 mmol) in MeCN (10 ml) was refluxed with stirring for 16 h. 0.26 ml (2.48 mmol) of 2-fluoronitrobenzene and 342.8 mg (2.48 mmol) of K₂CO3 was added to the reaction mixture and reflux was continued for 5 h. The reaction mixture was diluted with water (30 ml) and extracted with CH₂Cl₂ (40 ml×3). The extracts combined were dried (Na2SO4), filtered, and concentrated to give 1356 mg of crude product which was purified by silica gel column chromatography (n-hexane/acetone: 4/1) to afford 836 mg (92%) of title compound as yellow oil.

¹H NMR (270 MHz, CDCl₃) δ 8.32 (1H, br.s), 8.18 (1H, dd, J=1.5, 8.4 Hz), 7.47-7.39 (1H, m), 7.30-7.12 (4H, m), 6.91 (1H, br.d, J=8.4 Hz), 6.63 (1H, ddd, J=1.2, 7.2, 8.4 Hz), 3.46-3.37 (2H, m), 2.96-2.86 (4H, m), 2.53 (2H, t, J=6.8 Hz), 2.23-2.12 (2H, m), 2.07-1.88 (6H, m), 1.60-1.50 (2H, m).

### Example 18

### 2,3-Dihydro-1′-[3-(2-hydroxymethylbenzimidazol-1-yl)-3-oxopropyl]spiro [1H-indene-1,4′-piperidine]

To a stirred solution of nitroaniline derivative (836.3 mg, 2.29 mmol, this was prepared in preparation 11) in mixed solvent of MeOH (4.8 ml), THF (14.4 ml), and water (1.2 ml) was added NH4Cl (367 mg, 6.9 mmol) and Zn powder (1048 mg, 16 mmol) at 0° C. and resulting reaction mixture was stirred at room temperature for 1.5 h. After Celite filtration, the filtrate was concentrated. The resulting residue was diluted with aqueous NaHCO₃ solution (50 ml), extracted with CH₂Cl₂ (40 ml×4). The extracts combined were washed with brine, dried (Na₂SO₄), filtered, and concentrated to give 797.9 mg of crude phenylenediamine derivative as reddish brown oil.

¹H NMR (270 MHz, CDCl₃) δ 7.24-7.10 (4H, m), 6.88-6.63 (4H, m), 3.43 (1H, br.s), 3.22 (2H, t, J=6.3 Hz), 3.03-2.94 (2H, m), 2.90 (2H, t, J=7.4 Hz), 2.58 (2H, t, J=6.4 Hz), 2.24-2.11 (2H, m), 2.07-1.84 (8H, m), 1.62-1.50 (2H, m).

A mixture of this phenylenediamine derivative (50.3 mg, 0.15 mmol) and glycolic acid (22.8 mg, 0.3 mmol) in 4N HCl (3 ml) was refluxed with stirring for 22.5 h. After cool down to room temperature, the reaction mixture was basified with aqueous 25% NH₃ solution and extracted with CH₂Cl₂ (20 ml×3). The extracts combined were washed with water, dried (Na₂SO₄), filtered, and concentrated to give 51.6 mg of crude product, which was purified by preparative TLC (CH₂Cl₂/MeOH: 15/1, 3 times developed) to afford 25.8 mg of product. As this included some impurity, this was purified again by preparative TLC (AcOEt/i-PrOH/25% NH₃: 50/10/1) to give 18.8 mg (33%) of title product as pale yellow oil.

¹H NMR (270 MHz, CDCl₃) δ 7.79-7.70 (1H, m), 7.44-7.36 (1H, m), 7.31-7.15 (6H, m), 5.01 (2H, s), 4.48 (2H, t, J=6.3 Hz), 3.43 (1H, br.s), 2.87 (2H, t, J=7.3 Hz), 2.82-2.72 (2H, m), 2.34-1.89 (11H, m), 1.57-1.45 (2H, m).

This oil was converted to citric acid salt by mixing with 1 equivalent of citric acid in MeOH (1.5 ml) followed by concentration.

MS (ESI positive) m/z: 376 (M+H)⁺.

IR(KBr): 3396, 2937, 2600, 1717, 1589, 1458, 1209, 1045, 758 cm⁻¹

Anal. Calcd for C₂₄H₂₉N₃O—C₆H₈O₇-2H₂O: C, 59.69; H, 6.85; N, 6.96. Found: C, 59.90; H, 6.51; N, 6.56.

### Example 19

### 2,3-Dihydro-1′-[3-(2-hydroxymethylindolin-1-yl)-3-oxopropyl]spiro [1H-indene-1,4′-piperidine]

This was prepared according to the procedure described in Example 1 using 2-hydroxymethylindoline instead of methyl indoline 2-carboxylate. 126.3 mg (55.9%) of title compound as amorphous solid. This compound showed broadened spectra in proton NMR except for the following peaks.

¹H NMR (300 MHz, CDCl3) δ 2.89 (2H, t, J=7.3 Hz), 2.40-2.15 (2H, m), 2.05-1.80 (4H, m, including 2H, t, J=7.3 Hz at 2.00 ppm), 1.60-1.45 (2H, m).

This solid was converted to citric acid salt by mixing with 1 equivalent of citric acid in mixed solvent of CH₂Cl₂ and MeOH followed by concentration.

¹H NMR (270 MHz, DMSO-d6) δ 8.00 (1H, br.d, J=7.3 Hz), 7.30-7.12 (6H, m), 7.03 (1H, br.t, J=7.3 Hz), 4.70-4.55 (1H, m), 3.55-2.75 (14H, m, including 2H, t, J=7.1 Hz at 2.89 ppm), 2.63 (2H, d, J=15.2 Hz), 2.53 (2H, d, J=14.5 Hz), 2.13-1.95 (4H, m, including 2H, t, J =7.1 Hz at 2.06 ppm), 1.70-1.60 (2H, m).

MS (ESI positive) m/z: 391 (M+H)⁺.

IR(KBr): 3350, 2941, 2600,1728, 1641, 1595, 1481, 1420, 1211, 758 cm⁻¹

Anal. Calcd for C₂₅H₃₀N₂O₂—C₆H₈O₇-2H₂O: C, 60.18; H, 6.84; N, 4.53. Found: C, 60.52; H, 6.49; N, 4.49.

### Example 20

### 2,3-Dihydro-1′-[3-(2-methoxymethylindolin-1-yl)-3-oxopropyl]spiro [1H-indene-1,4′-piperidine]

To a stirred mixture of 2,3-dihydro-1′-[3-(2-hydroxymethylindolin-1-yl)-3-oxopropyl]spiro[1H-indene-1,4′-piperidine] (23.7 mg, 0.0607 mmol) and fluobolic acid (48% solution in water, 8.7 μl, 0.0668 mmol) in CH₂Cl₂ (2 ml) was added dropwise trimethylsilyldiazomethane (2M solution in hexane, 30.3 [t, 0.0668 mmol) at 0° C. and stirred for 1 h. Then fluobolic acid (48% solution in water, 8.7 μl, 0.0668 mmol) and trimethylsilyldiazomethane (2M solution in hexane, 30.3 μl, 0.0668 mmol) were added to the reaction mixture and stirred at room temperature for 1 h. The reaction mixture was quenched with water and extracted with CH₂Cl₂. The extracts combined were dried (Na₂SO₄), filtered, and concentrated. The residue was purified by preparative TLC (acetone/hexane: 1/1) to give 11.2 mg (45.5%) of title compound as an yellow oil.

¹H NMR (300 MHz, CDCl₃) δ 8.13 (1H, br.s), 7.25-7.12 (6H, m), 7.04 (1H, dd, J=7.5, 8.4 Hz), 4.65 (1H, br.s), 3.50-3.25 (5H, m, including 3H, s, at 3.31 ppm), 3.03-2.75 (10H, m, including 2H, t, J=7.3 Hz at 2.90 ppm), 2.36-2.24 (2H, m), 2.06-1.93 (4H, m, including 2H, t, J=7.3 Hz at 2.03 ppm), 1.63-1.54 (2H, m).

This was converted to HCl salt similar to that described in Example 1 to give 12.2 mg of HCl salt as a white solid.

MS (ESI positive) m/z: 405 (M+H)⁺.

IR(KBr): 3400, 2900, 2600, 1649, 1597, 1481, 1460, 1420, 1275, 1119, 758 cm⁻¹

### Example 21

### 2,3-Dihydro-1′-{3-[2-(S)-(2-hydroxyethyl)aminocarbonylindolin-1-yl]-3-oxopropyl}spiro[1H-indene-1,4′-piperidine] hydrochloride

This was prepared according to the procedure described in Example 17 using 2-hydroxyethylamine instead of N,N-dimethylethylenediamine and additionally DMF was added as solvent. Solvent ratio of CH₂Cl₂/THF/DMF was 2/2/1. 10.1 mg (30.4%) of title compound was obtained as amorphous solid.

¹H NMR (270 MHz, CDCl₃) δ 8.17 (1H, br.s), 7.26-6.80 (8H, m), 4.94 (1 H, br.s), 3.75-2.50 (15H, m), 2.45-2.20 (2H, m), 2.07-1.85 (4H, m, including 2H, t, J=7.1 Hz at 2.01 ppm), 1.63-1.50 (2H, m).

This was converted to HCl salt similar to that described in Example 1 to give 12.2 mg of HCl salt as a white solid.

MS (ESI positive) m/z: 448 (M+H)⁺.

IR(KBr): 3400, 2934, 2700, 1655, 1597, 1481, 1460, 1420, 1271, 1067, 758 cm⁻¹

### Example 22

### 2,3-Dihydro-1′-[3-(2-aminomethylindolin-1-yl)-3-oxopropyl]spiro[1H-indene-1,4′-piperidine]

A mixture of 2,3-dihydro-1′-[3-(2-hydroxymethylindolin-1-yl)-3-oxopropyl]spiro[1H-indene-1,4′-piperidine] (this was prepared in Example 19, 37.5 mg, 0.096 mmol), phthalimide (56.5 mg, 0.384mmol), N,N,N′N′-Atetramethylazodicarboxamide (66.1 mg, 0.384 mmol) and tributylphosphine (95.7 μl, 0.384 mmol) in THF (2 ml) was stirred at room temperature for 1 day. The reaction mixture was concentrated and the residue was purified by preparative TLC (1 mm thick plate×2, CH₂Cl₂/MeOH: 10:1) to give 106 mg of brown oil. This was purified again by preparative TLC (1 mm thick plate×2, AcOEt/i-PrOH/NH₃ solution in EtOH: 100/5/2) to give 57.5 mg of phthalimide derivative as brown oil. A mixture of this oil (57.5 mg) and hydrazine hydrate (18.7 μl, 0.384 mmol) in MeOH (3 ml) was refluxed with stirring for 4 h. After cool down to room temperature, the reaction mixture was concentrated. The resultant solid appeared was removed by filtration. The filtrate was concentrated and the residue was purified by silica gel column chromatography (EtOAc/hexane: 1/5) to give 13.1 mg (35%) of title compound.

¹H NMR (270 MHz, CDCl₃) δ 8.90-8.75 (1H, m), 7.25-6.95 (5H, m), 6.72-6.65 (1H, m), 6.60 (1H, d, J=7.8 Hz), 4.16-4.05 (1H, m), 3.52-3.45 (2H, m), 3.25-3.13 (1H, m), 2.95-2.75 (4H, m), 2.60-2.50 (2H, m), 2.42-2.35 (2H, m), 2.22-2.09 (2H, m), 1.99 (2H, t, J=7.4 Hz), 1.92-1.77 (2H, m), 1.63-1.35 (5H, m).

This was converted to HCl salt similar to that described in Example 1 to give 13.1 mg of HCl salt as a white solid.

MS (ESI positive) m/z: 390 (M+H)⁺.

IR(KBr): 3420, 3269, 2930, 2575, 2480, 1655, 1545, 1466, 1248, 756 cm−1

### Example 23

### 2,3-Dihydro-1′-{3-[2-(S)-(2-aminoethyl)aminocarbonylindolin-1-yl]-3-oxopropyl}spiro[1H-indene-1,4′-piperidine] dihydrochloride

This was prepared according to the procedure described in Example 21 using 2-t-butoxycarbonylaminoethylamine instead of 2-hydroxyethylamine followed by removal of Boc group by treatment of HCl solution in MeOH and basic workup. 18.1 mg (53.1%) of free base was obtained as white amorphous solid. This compound showed broadened spectra in proton NMR except for the following peaks.

¹H NMR (300 MHz, CDCl₃) δ 2.90 (2H, t, J=7.2 Hz), 2.01 (2H, t, J=7.3 Hz), 1.63-1.50 (2H, m).

This was converted to HCl salt similar to that described in Example 1 to give 18 mg of HCl salt as a white solid.

¹H NMR (300 MHz, DMSO-d6) δ 10.50 (1H, br.s), 8.75 (1H, br.s), 8.25-7.85 (4H, m, including 1H, d, J=7.9 Hz), 7.35-7.00 (7H, m), 5.20-5.12 (1H, m), 3.75-2.70 (16H, m), 2.35-2.15 (2H, m), 2.09 (2H, t, J=7.2 Hz), 1.73-1.62 (2H, m).

MS (ESI positive) m/z: 447 (M+H)⁺.

IR(KBr): 3400, 3236, 2941, 2700, 2575, 1655, 1597, 1541, 1481, 1462, 1416, 1269, 970, 758 cm⁻¹.

Anal. Calcd for C₂₇H₃₄N₄O₂-2HCl-2.9H₂O: C, 56.72; H, 7.37; N, 9.80. Found: C, 56.97; H, 7.35; N, 9.75.

### Example 24

### 2,3-Dihydro-1′-{3-[2-(S)-(2-acetamidoethyl)aminocarbonylindolin-1-yl]-3-oxopropyl}spiro[1H-indene-1,4′-piperidine] hydrochloride

A mixture of 2,3-dihydro-1′-{3-[2-(S)-(2-aminoethyl) aminocarbonyllindolin-1yl]-3-oxopropyl}spiro[1H-indene-1,4′-piperidine] (this was prepared in Example 23, 55 mg, 0.053 mmol), acetic anhydride (15.1 μl, 0.16 mmol), and 4-dimethylaminopyridine (1.3 mg, 0.011 mmol) in pyridine (3 ml) was stirred at room temperature for 4 h. After evaporation of the pyridine, the residue was diluted with 2N HCl and CH₂Cl₂. The mixture was extracted with CH₂Cl₂. The extracts combined were washed with saturated aqueous NaHCO₃ solution and brine, dried (Na₂SO₄), filtered, and concentrated. The residue was purified by preparative TLC (CH₂Cl₂/MeOH:10/1) to give 23.2 mg (89.2%) of free base as amorphous solid. This compound showed broadened spectra in proton NMR except for the following peaks.

¹H NMR (270 MHz, CDCl₃) δ 7.06 (1H, dd, J=7.0, 7.3 Hz), 2.92 (2H, t, J=7.4 Hz), 2.03 (2H, t, J=7.4 Hz), 1.75-1.50 (2H, m).

This was converted to HCl salt similar to that described in Example 1 to give 23 mg of HCl salt as a white solid.

¹H NMR (300 MHz, DMSO-d6) δ 8.52 (1H, br.s), 8.08 (1H, d, J=7.9 Hz), 7.30-6.95 (8H, m), 5.13-5.05 (1H, m), 3.65-2.45 (17H, m), 2.30-2.00 (4H, m), 1.82 (3H, s), 1.75-1.60 (2H, m).

MS (ESI positive) m/z: 489 (M+H)⁺.

IR(KBr): 3400, 3267, 2936, 2700, 2573, 1655, 1545, 1481, 1416, 1246, 746 cm⁻¹.

Anal. Calcd for C₂₉H₃₆N₄O₃—HCl-2.2H₂O: C, 61.68; H, 7.39; N, 9.92. Found: C, 61.60; H, 7.33; N, 9.89.

### Example 25

### 2,3-Dihydro-1′-{3-[2-(S)-(2-methanesulfonamidoethyl) aminocarbonylindolin-1-yl]-3-oxopropyl}spiro[1H-indene-1,4′-piperidine] hydrochloride

A mixture of 2,3-dihydro-1′-{3-[2-(S)-(2-aminoethyl) aminocarbonylindolin-1-yl]-3-oxopropyl}spiro[1H-indene-1,4′-piperidine] (this was prepared in Example 23, 55.2 mg, 0.052 mmol), mesyl chloride (6 μl, 0.077 mmol), and triethylamine (21.6 μl, 0.155 mmol) in CH₂C]₂ (2 ml) was stirred at room temperature for 1 day. The reaction mixture was diluted with saturated NaHCO₃ aqueous solution and extracted with CH₂Cl₂. The extracts combined were dried (Na₂SO₄), filtered, and concentrated. The residue was purified by preparative TLC (CH₂Cl₂/MeOH:10/1) to give 10.5 mg (38.7%) of free base as amorphous solid. This compound showed broadened spectra in proton NMR except for the following peaks.

¹H NMR (270 MHz, CDCl₃) δ 7.06 (1H, dd, J=7.3, 7.8 Hz), 2.90 (3H, s), 2.03 (2H, t, J=7.4 Hz), 1.75-1.50 (2H, m).

This was converted to HCl salt similar to that described in Example 1 to give 10.5 mg of HCl salt as a white solid.

¹H NMR (300 MHz, CDCl₃) δ 10.22 (1H, br.s), 8.15 (1H, d, J=7.2 Hz), 7.90-7.00 (10H, m), 5.30-5.05 (1H, m), 4.30-2.85 (17H, m, including 3H, s, at 2.96 ppm), 2.75-2.45 (2H, m), 2.40-1.90 (3H, m), 1.85-1.65 (2H, m).

MS (ESI positive) m/z: 525 (M+H)⁺.

IR(KBr): 3400, 2936, 2700, 2573, 1655, 1483, 1313, 1151, 758 cm⁻¹.

### Preparation 12

### Methyl 2-(Benzothiazol-2-one-1-yl)-4-hydroxybutyrate

To a stirred solution of 2-hydroxybenzothiazole (300 mg, 1.98 mmol) in DMF (5 ml) was added NaH (60% oil suspension, 160 mg, 3.97 mmol) at room temperature. To this mixture was added (α-bromo-γ-butyrolactone (660 mg, 3.97 mmol) and resulting reaction mixture was stirred at room temperature for 1 h, and at 60° C. for 30 minutes. Then NaH (80 mg, 1.98 mmol) and α-bromo-γ-butyrolactone (330 mg, 1.98 mmol) was added to the reaction mixture and stirred at 60° C. for 1 h. The reaction mixture was poured into aqueous NaHCO₃ solution and extracted with ethyl acetate. The extracts combined were dried (MgSO₄) and concentrated. The residue was purified by silica gel column chromatography (hexane/ethyl acetate: 3/2) to give 0.35 g (75%) of lactone derivative as white solid.

¹H NMR (300 MHz, CDCl₃) δ 7.47 (1H, dd, J=0.9, 7.6 Hz), 7.32 (1H, ddd, J=1.3, 7.5, 7.7 Hz), 7.20 (1H, ddd, J=1.1, 7.7, 7.7 Hz), 6.93 (1H, d, J=8.0 Hz), 5.45-5.30 (1H, m), 4.71 (1H, ddd, J=2.4, 9.2, 9.3 Hz), 4.46 (1H, ddd, J=7.0, 9.3, 10.1 Hz), 2.88-2.62 (2H, m).

To a stirred suspension of the above lactone derivative (0.39 g, 1.66 mmol) in MeOH (12 ml) was added c-H₂SO₄ (1 ml) and the reaction mixture was stirred at 60° C. for 2 h. The reaction mixture was poured into water and extracted with ethyl acetate. The extracts combined were washed with aqueous NaHCO₃ solution and brine, dried (MgSO₄), filtered, and concentrated. The residue was purified by silica gel column chromatography (CH₂Cl₂/MeOH: 10/1) followed by preparative TLC (1 mm thick plate, CH₂Cl₂/MeOH: 20/1) to give 173 mg (39%) of the title compound as a colorless oil.

¹H NMR (270 MHz, CDCl₃) δ 7.48 (1H, dd, J=1.3, 7.7 Hz), 7.30 (1H, ddd, J=1.5, 7.7, 7.9 Hz), 7.19 (1H, ddd, J=1.1, 7.6, 7.7 Hz), 7.00 (1H, d, J=7.9 Hz), 5.47 (1H, dd, J=4.6, 10.7 Hz), 3.80-3.74 (1H, m), 3.74 (3H, s), 3.50-3.40 (1H, m), 2.67-2.53 (1H, m), 2.35-2.22 (1H, m), 2.06-1.97 (1H, m).

### Preparation 13

### 2,3-Dihydro-1′-[3-(benzothiazol-2-one-1-yl)-3-methoxycarbonylpropyl]spiro[1H-indene-1,4′-piperidine]

To a stirred solution of methyl 2-(benzothiazol-2-one-1-yl)-4-hydroxybutyrate (0.21 g, 0.79 mmol) and triethylamine (0.14 ml, 1.03 mmol) in CH₂Cl₂ (5 ml) was added mesyl chloride (67 μl, 0.86 mmol) at 0° C. After 15 min stirring, the reaction mixture was poured into aqueous NaHCO₃ solution and extracted with CH₂Cl₂. The extracts combined were dried (MgSO₄), filtered, and concentrated. To this residue was added toluene and concentrated again to give 0.30 g of crude mesylate as colorless oil.

¹H NMR (270 MHz, CDCl₃) δ 7.47 (1H, br.d, J=7.7 Hz), 7.35-7.15 (2H, m), 7.19 (1H, br.d, J=8.2 Hz), 5.37-5.27 (1H, m), 4.45-4.35 (1H, m), 4.17-4.07 (1H, m), 3.75 (3H, s), 2.94 (3H, s), 2.90-2.78 (1H, m), 2.65-2.50 (1H, m).

A mixture of this oil (0.30 g, 0.79 mmol), 2,3-dihydrospiro[1H-indene-1,4′-piperidine] hydrochloride (0.194 g, 0.87 mmol), and diisopropylethylamine (0.31 g, 2.37 mmol) in MeOH (10 ml) was stirred at 60° C. for 14 h and at 80° C. for 4 h. The reaction mixture was concentrated, then diluted with CH₂Cl₂, washed with aqueous NaHCO₃ solution, dried (MgSO₄), filtered, and concentrated. The residue was purified by silica gel column chromatography (CH₂Cl₂/MeOH: 30/1) to give 165 mg (48%) of title compound as colorless oil.

¹H NMR (270 MHz, CDCl₃) δ 7.45 (1H, dd, J=1.6, 8.2 Hz), 7.33-7.26 (1H, m), 7.22-7.12 (6H, m), 5.47-5.36 (1H, m), 3.74 (3H, s), 2.90-2.82 (3H, m, including 2H, t, J=7.1 Hz at 2.86 ppm), 2.65-2.50 (2H, m), 2.42-2.25 (3H, m), 2.15-2.05 (2H, m), 1.95 (2H, t, J=7.3 Hz), 1.92-1.65 (2H, m), 1.60-1.37 (2H, m).

### Example 26

### 2,3-Dihydro-1′-[3-(benzothiazol-2-one-1-yl)-3-hydroxymethylpropyl]spiro[1 H-indene-1,4′-piperidine]

To a stirred solution of 2,3-dihydro-1′-[3-(benzothiazol-2-one-1-yl)-3-methoxycarbonyl-propyl]spiro[1H-indene-1,4′-piperidine] (40 mg, 0.092 mmol) in THF (2 ml) was added LiAIH₄ (3.5 mg, 0.092 mmol) at 0° C. After 30 min stirring, LiAIH₄ (7 mg, 0.184 mmol) was added to the reaction mixture and stirring was continued another 10 min at 0° C. The reaction mixture was quenched with 15 μl of water, 15 μl of 2N NaOH solution, and 45 μl of water, then the resulting mixture was stirred for 20 min at room temperature. After Celite filtration, the filtrate was concentrated. The residue was purified by preparative TLC (CH₂Cl₂/MeOH: 10/1, then ethyl acetate) to give 8 mg (22%) of title compound as white solid.

¹H NMR (270 MHz, CDCl₃) δ 7.44-7.40 (1H, m), 7.34-7.30 (2H, m), 7.24-7.12 (6H, m), 4.65-4.40 (1H, m), 4.20 (1H, dd, J=6.4, 11.7 Hz), 3.95 (1H, dd, J=7.6, 11.8 Hz), 3.16-3.02 (1H, m), 2.90 (2H, t, J=7.2 Hz), 2.85-2.75 (1H, m), 2.62-2.48 (3H, m), 2.39-2.26 (1H, m), 2.20-2.08 (1H, m), 2.08-1.84 (5H, m, including 2H, t, J=7.4 Hz at 2.00 ppm), 1.65-1.50 (2H, m).

This was treated with HCl solution in MeOH followed by concentration to give 8 mg of HCl salt as white amorphous solid.

MS (ESI positive) m/z: 409 (M+H)⁺.

### Preparation 14

### 2,3-Dihydro-1′-[3-(benzothiazol-2-one-1-yl)-3-carboxypropyl]spiro[1H-indene-1,4′-piperidine]

A mixture of 2,3-dihydro-1′-[3-(benzothiazol-2-one-1-yl)-3-methoxycarbonylpropyl] spiro[1H-indene-1,4′-piperidine] (110 mg, 0.25 mmol) and 2N NaOH solution (0.5 ml, 1 mmol) in THF (2 ml) and MeOH (1 ml) was stirred at room temperature for 16 h. The reaction mixture was diluted with ethyl acetate, washed with HCl solution and brine, dried (MgSO₄), filtered, and concentrated to give 103 mg (96%) of title compound as white solid.

¹H NMR (300 MHz, DMSO-d6) δ 7.73 (1H, d, J=7.9 Hz), 7.46−7.36 (2H, m), 7.30−7.05 (5H, m), 5.45−5.35 (1H, m), 3.55−2.95 (9H, m), 2.86 (2H, t, J=7.1 Hz), 2.80−2.63 (1H, m), 2.25−1.95 (4H, m, including 2H, t, J=7.5 Hz at 2.02 ppm), 1.70−1.56 (2H, m).

MS(El direct) m/z: 422(M)⁺.

### Example 27

### 2,3-Dihydro-1′-[3-(benzothiazol-2-one-1-yl)-3-(N,N-dimethylaminocarbonyl)propyl]spiro[1H-indene-1,4′-piperidine]

This was prepared according to the procedure described in Example 11 using 2,3-dihydro-1′-[3-(benzothiazol-2-one-1-yl)-3-carboxypropyl]spiro[1H-indene-1,4′-piperidine] instead of 2,3-dihydro-1′-[3-(2-carboxyindolin-1-yl)-3-oxopropyl]spiro[1H-indene-1,4′-piperidine]. Yield was 30 mg (71%). Product was colorless amorphous solid.

¹H NMR (270 MHz, CDCl₃) δ 7.55−7.49 (1H, m), 7.46−7.41 (1H, m), 7.30−7.09 (6H, m), 5.72−5.62 (1H, m), 2.96 (3H, s), 2.95 (3H, s), 2.88−2.73 (4H, m, including 2H, t, J=7.2 Hz at 2.85 ppm), 2.50−2.22 (4H, m), 2.20−1.80 (5H, m, including 2H, t, J=7.4 Hz at 1.93 ppm), 1.70−1.55 (1H, m), 1.50−1.35 (2H, m).

This was treated with HCl solution in MeOH followed by concentration to give 30 mg of HCl salt as white amorphous solid.

MS (ESI positive) m/z: 450 (M+H)⁺.

IR(KBr): 3439, 2932, 2563, 1655,1589, 1472, 758 cm⁻¹

Anal. Calcd for C₂₆H₃₁N₃O₂S—HCl—H₂O: C, 61.95; H, 6.80; N, 8.34. Found: C, 62.33; H, 7.00; N, 7.89.

### Example 28

### 2,3-Dihydro-1′-[3-(benzothiazol-2-one-1-yl)-3-(2-N,N-dimethylaminoethylaminocarbonylpropyl]spiro[1H-indene-1,4′-piperidine]

This was prepared according to the procedure described in Example 27 using N,N-dimethylethylenediamine instead of dimethylamine hydrochloride. Yield was 30 mg (80%). Product was colorless oil.

¹H NMR (270 MHz, CDCl3) δ 7.45 (1H, br.d, J=7.7 Hz), 7.32−7.10 (7H, m), 6.77 (1H, br.s), 5.41 (1H, dd, J=5.3, 9.0 Hz), 3.40−3.20 (2H, m), 2.90−2.75 (3H, m, including 2H, t, J=7.4 Hz at 2.85 ppm), 2.70−2.50 (2H, m), 2.45−1.75 (16H, m, including 6H, s at 2.05 ppm and 2H, t, J=7.2 Hz at 1.93 ppm), 1.70−1.30 (3H, m).

This was treated with HCl solution in MeOH followed by concentration to give 32 mg of HCl salt as white amorphous solid.

MS (ESI positive) m/z: 493 (M+H)⁺.

IR(KBr) : 3408, 2934, 2691, 1670,1537, 1472, 758 cm⁻¹

Anal. Calcd for C₂₈H₃₆N₄O₂S-2HCl-1.2H₂O: C, 57.27; H, 6.93; N, 9.54. Found: C, 57.623; H, 7.31; N, 9.07.

### Example 29

### 2,3-Dihydro-1′-[3-(3-ethylbenzimidazol-2-one-1-yl)propyl]spiro[1H-indene-1,4′-piperidine]

NaH (60% oil suspension, 11.7 mg, 0.293 mmol) was washed with hexane (2 ml×2) and decanted, then DMF (1 ml) was added. To a stirred this suspension was added a solution of 2,3-dihydro-1′-[3-(benzimidazol-2-one-1-yl)propyl]spiro[1H-indene-1,4′-piperidin] (66.1 mg, 0.193 mmol) in DMF (1.5 ml) at room temperature. After stirring for 0.5 h, a solution of iodoethane (57.1 mg, 0.366 mmol) was dropwisely added to the reaction mixture at 0° C. and the resulting mixture was stirred at room temperature for 19 h. The reaction mixture was diluted with saturated aqueous NaHCO₃ solution and extracted with ethyl acetate. The extracts combined were washed with water, dried (Na₂SO₄), filtered, and concentrated to give 67.5 mg of crude product, which was purified by preparative TLC (CH₂Cl₂/MeOH: 15/1) to give 30.5 mg (43%) of title compound as pale yellow oil.

¹H NMR (270 MHz, CDCl₃) δ 7.25−6.98 (8H, m), 4.01−3.91 (4H, m), 2.92−2.82 (4H, m), 2.46 (2H, t, J=6.9 Hz), 2.20−2.07 (2H, m), 2.06−1.76 (6H, m), 1.58−1.48 (2H, m), 1.35 (3H, t, J=7.2 Hz).

This was converted to citric acid salt according to the procedure described in Example 34 to give 38.3 mg of citrate as white amorphous solid.

MS (ESI positive) m/z: 390 (M+H)⁺.

IR(KBr): 3416, 2937, 2584, 1686, 1492, 1420,1192, 756 cm⁻¹

Anal. Calcd for C₂₅H₃₁N₃O—C₆H₈O₇-1.2H₂O: C, 61.72; H, 6.92; N, 6.97. Found: C, 61.83; H, 6.94; N, 6.51.

### Example 30

### 2,3-Dihydro-1′-[3-(2-acetamidobenzimidazol-1-yl)propyl]spiro[1H-indene-1,4′-piperidine]

To a stirred solution of 2,3-Dihydro-1′-[3-(2-aminoanilino)propyl]spiro[1H-indene-1,4′-piperidine] (this was prepared in the first step of Example 18, 105.7 mg, 0.315 mmol) in THF (1 ml) was added a solution of cyanogen bromide (33.4 mg, 0.315 mmol) in mixed solvent of THF (1 ml) and water (1 ml) at room temperature. After 16.5 h, the reaction mixture was basified by 25% NH3 solution in water at ° C. and extracted with CH₂Cl₂. The extracts combined were dried (Na₂SO₄), filtered, and concentrated to give 114.3 mg of crude product. To a solution of this compound (53.1 mg, 0.147 mmol) in CH₂Cl₂ (1.5 ml) was added catalytic amount of 4-dimethylaminopyridine, triethylamine (41 μl, 0.726 mmol), and a solution of acetyl chloride (17.3 mg, 0.221 mmol) in CH₂Cl₂ (1.5 ml) at 0° C. After 2 h stirring, the reaction mixture was warmed to room temperature and stirred another 3 h. The reaction mixture was quenched with saturated aqueous NaHCO₃ solution (10 ml) and extracted with CH₂Cl₂. The extracts combined were washed with brine, dried (Na₂SO₄), filtered, and concentrated. The residue was purified by preparative TLC (CH₂Cl₂/MeOH: 15/1) to afford 7.6 mg (13%) of title compound as pale yellow oil.

¹H NMR (270 MHz, CDCl₃) δ 7.35−7.10 (8H, m), 4.25−4.15 (4H, m), 2.96−2.82 (8H, m), 2.22−1.96 (7H, m, including 3H, s, at 2.17 ppm), 1.75−1.50 (3H, m).

MS (EI direct) m/z: 402 (M⁺), 227, 189.

This was converted to citric acid salt according to the procedure described in Example 34 to give 4.6 mg of citrate as white amorphous solid.

Anal. Calcd for C₂₅H₃₀N₄O—C₆H₈O₇-1.5H₂O: C, 59.89; H, 6.65; N, 9.01. Found: C, 60.15; H, 6.58; N, 8.76.

### Example 31

### 2,3-Dihydro-1′-{3-[3-(2-hydroxyethyl)benzimidazol-2-one-1-yl)propyl}spiro[1H-indene-1,4′-piperidine]

This was prepared according to the procedure described in Example 29 using t-butyldimethylsilyloxyethyl bromide instead of iodoethane followed by deprotection using tetrabutylammonium fluoride in THF. Yield was 48.4 mg (57%). Product was colorless oil.

¹H NMR (270 MHz, CDCl₃) δ 7.23−6.99 (8H, m), 4.09−3.92 (6H, m), 2.92−2.80 (4H, m, including 2H, t, J=7.2 Hz), 2.45 (2H, t, J=7.1 Hz), 2.19−2.07 (2H, m), 2.05−1.83 (6H, m), 1.75 (1H, br.s), 1.58−1.46 (2H, m).

MS (El direct) m/z: 405 (M⁺), 375, 275, 200.

This was converted to citric acid salt according to the procedure described in Example 34 to give 11.6 mg of citrate as white amorphous solid.

IR(KBr): 3406, 2939, 2579, 1686, 1495, 1416, 1192, 756 cm⁻¹

Anal. Calcd for C₂₅H₃₁N₃O₂—C₆H₈O₇-2H₂O: C, 58.76; H, 6.84; N, 6.63. Found C, 58.93; H, 6.62; N, 6.33

### Example 32

### 2,3-Dihydro-1′-{3-[3-(2-aminoethyl)benzimidazol-2-one-1-yl)propyl}spiro[1H-indene-1,4′-piperidine]

This was prepared according to the procedure described in Example 29 using N-(2-bromoethyl)phthalimide instead of iodoethane followed by deprotection using hydrazine hydrate in MeOH. Yield was 20.1 mg (10.8%). Product was colorless oil.

¹H NMR (270 MHz, CDCl₃) δ 7.23−7.02 (8H, m), 4.02−3.92 (4H, m), 3.08 (2H, t, J=6.2 Hz), 2.92−2.80 (4H, m, including 2H, t, J=7.4 Hz at 2.88 ppm), 2.46 (2H, t, J=6.9 Hz), 2.20−2.07 (2H, m), 2.06−1.83 (6H, m), 1.58−1.48 (2H, m), 1.26 (2H, br.s),.

MS (EI direct) m/z: 404 (M⁺), 277, 200.

This was converted to citric acid salt according to the procedure described in Example 34 to give 7.5 mg of citrate as white amorphous solid.

Anal. Calcd for C₂₅H₃₂N₄O—C₆H₈O₇-3H₂O: C, 57.22; H, 7.13; N, 8.6 Found: C, 57.35; H, 6.82; N, 8.45.

### Example 33

### 2,3-Dihydro-1′-{3-[3-(2-acetamidoethyl)benzimidazol-2-one-1-yl)propyl}spiro[1H-indene-1,4′-piperidine]

To a stirred solution of 2,3-dihydro-1′-{3-[3-(2-aminoethyl)benzimidazol-2-one-1-yl)propyl}spiro[1H-indene-1,4′-piperidine] (12.7 mg, 0.031 mmol, this was prepared in Example 32) in CH₂Cl₂ (1.5 ml) was added catalytic amount of 4-dimethylaminopyridine and triethylamine (7.9 μl, 0.056 mmol) followed by addition of acetyl chloride (2.6 μl, 0.037 mmol) at 0° C. After 1 h stirring at 0° C. and 2 h stirring at room temperature, acetyl chloride (2.6 μl, 0.037 mmol) and triethylamine (7.9 μl, 0.056 mmol) were added to the reaction mixture at 0° C. After 1 h stirring at 0° C. and 2 h stirring at room temperature, the reaction mixture was quenched with saturated aqueous NaHCO₃ solution and extracted with CH₂Cl₂. The extracts combined were washed with brine, dried (Na₂SO₄), and concentrated to give 14 mg of crude product, which was purified by preparative TLC (CH₂Cl₂/MeOH: 10/1) to afford 12.5 mg (90%) of title compound as colorless oil.

¹H NMR (270 MHz, CDCl₃) δ 8 7.24−7.02 (8H, m), 6.40 (1H, br.s), 4.07 (2H, t, J=5.6 Hz), 3.98 (2H, t, J=6.9 Hz), 3.64−3.55 (2H, m), 2.92−2.80 (4H, m, including 2H, t, J=7.3 Hz at 2.89 ppm), 2.46 (2H, t, J=6.8 Hz), 2.20−2.07 (2H, m), 2.05−1.83 (9H, m, including 3H, s, at 1.95 ppm), 1.60−1.46 (2H, m).

This was converted to citric acid salt according to the procedure described in Example 34 to give 8.7 mg of citrate as white amorphous solid.

MS (ESI positive) m/z: 447 (M+H)⁺.

IR(KBr): 3400, 2943, 2579, 1690, 1495, 1418,1198, 754 cm⁻¹

Anal. Calcd for C₂₇H₃₄N₄O₂—C₆H₈O₇-1.9H₂O: C, 58.90; H, 6.86; N, 8.33. Found: C, 59.22; H, 6.57; N, 7.93

### Example 34

### 2,3-Dihydro-1′-[3-(2-(S)-N-methylaminocarbonylindolin-1-yl)-3-oxopropyl]spiro[1H-indene-1,4′-piperidine] citrate

This was prepared according to the procedure described in Example 11 using N-methylamine hydrochloride instead of dimethylamine hydrochloride. Yield was 32 mg (62%). Product was colorless amorphous solid. This compound showed broadened spectra in proton NMR except for the following peaks.

¹H NMR (270 MHz, CDCl₃) δ 5 2.79 (3H, d, J=4.8 Hz), 2.35−2.20 (2H, m), 2.05−1.85 (4H, m), 1.62−1.50 (2H, m).

This was dissolved in mixed solvent of CH₂Cl₂ (1 ml) and MeOH (1 ml) followed by addition of citric acid (15 mg, 0.0766 mmol) and resulting mixture was stirred for 2 h. After concentration, the residue was solidified by adding CH₂Cl₂-hexane. The resulting solid was collected by filtration and washed with ether to give 37 mg of citrate as white amorphous solid.

MS (ESI positive) m/z: 418 (M+H)⁺.

IR(KBr): 3362, 2937,2586,1728, 1653, 1597, 1483,1411, 758 cm⁻¹

Anal. Calcd for C₂₆H₃₁N₃O₂—C₆H₈O₇-2.3H₂O: C, 59.03; H, 6.75; N, 6.45. Found C, 59.41; H, 6.49; N, 5.87

### Example 35

### 2,3-Dihydro-1′-[3-(2-(S)-N,N-dimethylaminocarbonylindolin-1-yl)-3-oxopropyl]spiro[1H-indene-1,4′-piperidine] citrate

This was prepared according to the procedure described in Example 11. Yield was 24 mg (45%). Product was colorless amorphous solid.

¹H NMR (270 MHz, CDCl₃) δ 8.30 (0.4H, br.d, J=8.2 Hz), 7.32−7.08 (6.6H, m), 7.03−6.96 (1H, m), 5.54−5.42 (0.6H, m), 5.33−5.21 (0.4H, m), 3.77−3.60 (0.4H, m), 3.55−3.38 (0.6H, m), 3.03−2.80 (14H, m, including 1.2H, s, at 3.00 ppm, 1.8H, s, at 2.98 ppm, 1.2H, s, at 2.93 ppm, and 1.8H, s, at 2.90 ppm), 2.70−2.20 (3H, m), 2.10−1.90 (4H, m), 1.65−1.50 (2H, m).

This was converted to citric acid salt according to the procedure described in Example 34 to give 30 mg of citrate as white amorphous solid.

MS (ESI positive) m/z: 432 (M+H)⁺.

IR(KBr): 3416, 2936, 2561, 1728, 1655, 1597, 1485, 1406, 758 cm⁻¹

Anal. Calcd for C₂₇H₃₃N₃O₂—C₆H₈O₇—H₂O: C, 61.77; H, 6.75; N, 6.55. Found: C, 61.96; H, 6.84; N, 6.24

### Example 36

### 2,3-Dihydro-1′-{3-[2-(S)-(4-morpholinecarbonyl)indolin-1-yl]-3-oxopropyl}spiro[1H-indene-1,4′-piperidine] citrate

This was prepared according to the procedure described in Example 11 using morpholine instead of dimethylamine hydrochloride. Yield was 37 mg (63%). Product was colorless amorphous solid.

1H NMR (270 MHz, CDCl₃) δ 6 8.29 (0.4H, br.d, J=8.0 Hz), 7.35−6.96 (7.6H, m), 5.50−5.30 (1H, m), 3.90−3.40 (10H, m), 3.20−2.70 (8H, m), 2.65−2.20 (3H, m), 1.68−1.50 (2H, m).

This was converted to citric acid salt according to the procedure described in Example 34 to give 45 mg of citrate as white amorphous solid.

MS (ESI positive) m/z: 474 (M+H)⁺.

IR(KBr): 3414, 2930, 2573, 1728, 1655, 1597, 1485,1437, 1236, 1115, 758 cm⁻¹

Anal. Calcd for C₂₉H₃₅N₃O₃—C₆H₈O₇-1.5H₂O: C, 60.68; H, 6.69; N, 6.07. Found: C, 60.62; H, 6.66; N, 5.71

### Example 37

### 2,3-Dihydro-1′-[3-(2-(S)-aminocarbonylindolin-1-yl)-3-oxopropyl]spiro[1H-indene-1,4′-piperidine] hydrochloride

This was prepared according to the procedure described in Example 14 using (2S)-indolinecarboxamide instead of methyl (2S)-indolinecarboxylate. Yield was 82 mg (59%). Product was pale brown amorphous solid. This compound showed broadened spectra in proton NMR except for the following peaks.

¹H NMR (270 MHz, CDCl₃) δ 2.40−2.20 (2H, m), 2.10−1.85 (4H, m), 1.75−1.50 (2H, m).

This was converted to HCl salt according to the procedure described in Example 1 to give 60 mg of hydrochloride as pale brown amorphous solid.

MS (ESI positive) m/z: 404 (M+H)⁺.

IR(KBr): 3385, 2936, 2569, 1684, 1655, 1597, 1481, 1420, 1271, 756 cm⁻¹

Anal. Calcd for C₂₅H₂₉N₃O₂—C₆H₈O₇-1.2H₂O: C, 65.05; H, 7.07; N, 9.10. Found: C, 65.09; H, 7.17; N, 8.93

The title compound of Example 9 showed lC₅₀ values of 89 nM for ORL-1 receptor and 871 nM for mu-receptor respectively. This compound showed selective affinity for ORL-1 receptor over mu-receptor.

1. A compound of the following formula: 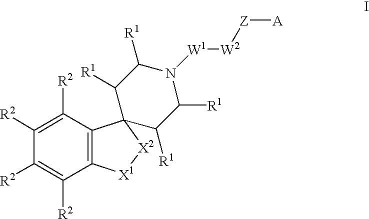
or salts thereof, wherein
each R¹ is independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or two R¹ groups taken together form —CH₂— or —(CH₂)₂— and the remaining R¹ groups are defined as above;
each R² is independently selected from
hydrogen;
halo;
hydroxy;
(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3}and R^{a4} are indendently selected from hydrogen, (C₁-C₆)alkyl, [(C₁-C₆)alkyl]-C(═O)—, [(C₁C₆)alkoxy]-C(═O)— and [(C₁-C₆)alkyl]-SO₂—;
(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R⁶, R^{a7} and R^{a8} are independently selected from hydrogen, (C₁C₆)alkyl, [(C₁-C₆)alkyl]-C(═O)—, [(C₁-C₆)alkoxy]-C(═O)— and [(C₁-C₆)alkyl]—SO₂—;
non-, mono- and di-substituted amino wherein the substituents are independently selected from (C₁C₈)alkyl, [(C₁-C₆)alkyl]-C(═O)—, [(C₁-C6)alkoxy ]-C(═O)— and [(C₁-C₆)alkyl]-SO₂—;
aryl selected from phenyl and naphthyl; and
four- to eight-membered heterocyclyl containing one to four hetero atoms in the ring independently selected from nitrogen, oxygen and sulfur;
X¹ and X² are independently selected from
(CH₂)ₙ₁ wherein n1 is an integer selected from 1, 2 and 3;
O;
NH;
S;
C(═O);
SO₂;
NR^{X1};
N—C(═O)R^{X2} ;
N—C(═O)OR^{X3}; and
N—C(═O)NR^{X4}R^{X5};
wherein
R^{X1}, R^{X2}, R^{X3}, R^{X4} and R^{X5} are independently (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or
X¹ and X² taken together form CH═CH;
W¹ and W² are independently selected from CR^{W1}R^{W2},
wherein
R^{W1} and R^{W2} are independently selected from
hydrogen;
halo;
hydroxy;
(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;
(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;
C(═O)—[(C₁-C₆)alkyl] wherein said (C₁-C₆)alkyl is optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1},R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;
C(═O)—NR^{W11}R^{W12} wherein R^{W11} and R^{W12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;
NR^{W13}R^{W14} wherein R^{W13} and R^{W14} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;
aryl selected from phenyl and naphthyl; and
four- to eight-membered heterocyclyl containing one to four hetero atoms independently selected from nitrogen, oxygen and sulfur;
A is selected from AA; AB; AC and AD: 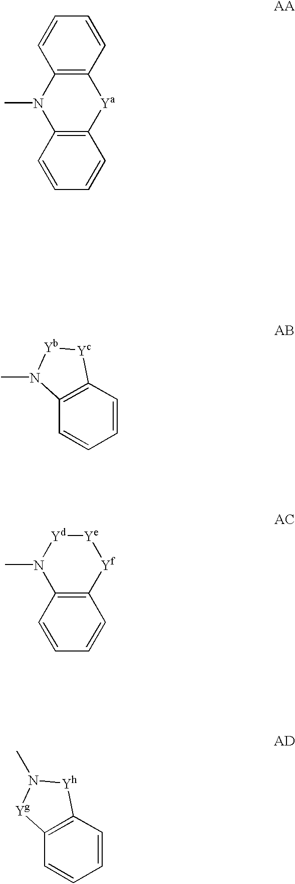
wherein Y^{a} is selected from (CH₂)ₙ₂ wherein n2 is an integer selected from 0, 1 and 2; C(═O); NH; O and S;
Y^{b}, Y^{c}, Y^{d}, Y^{e}, Y^{f}, Y^{g} and Y^{h} and Y^{h} are independently selected from
C(═O);
CR^{Y1} R^{Y2};
CR^{Y3}[C(═O)R^{Y4}];
CR^{Y3}[NR^{Y5}C(═O)R^{Y4}];
CR^{Y3}C(═O)NR^{Y6}R^{Y7};
CR^{Y3}[NR^{Y6}RY7];
O;
S;
SO₂;
NH;
N[(C₁-C₆)alkyl] wherein said (C₁-C₆)alkyl is optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;
N—(CH₂)ₙ₃-heterocyclyl wherein n3 is an integer selected from 0, 1, 2 and 3, and said heterocyclyl contains from four to eight ring atoms one or two of which are independently selected from nitrogen, oxygen and sulfur;
N—(CH₂)ₙ₄-aryl wherein n4 is an integer selected from 0, 1, 2 and 3, and said aryl is selected from phenyl and naphthyl; and
N—(CH₂)ₙ₅-heteroaryl wherein n5 is an integer selected from 0, 1, 2 and 3, and said heteroaryl is a five to ten membered aromatic heterocyclyl containing from one to four hetero atoms independently selected from nitrogen, oxygen and sulfur; or
Y^{b} and Y^{c} taken together form a group selected from CR^{Y81}═CR^{Y82}; CR^{Y83}═N and N═N; and Y^{d}, Y^{e}, Y^{f}, Y^{g} and Y^{h} are defined as above; wherein
R^{Y1}, R^{Y2} and R^{Y5} are independently selected from
hydrogen;
hydroxy;
non-, mono- and di-substituted amino wherein the substituents are independently selected from (C₁-C₆)alkyl; [(C₁-C₆)alkyl]-C(═O)—; [(C₁-C₆)alkoxy]-C(═O)—; [(C₁-C₆)alkyl]-SO₂—; and four- to eight-membered heterocyclyl containing one to four hetero atoms independently selected from nitrogen, oxygen and sulfur, wherein said heterocyclyl is optionally substituted with one to three substituents independently selected from hydroxy, (C₁-C₆)alkyl, NH₂—C(O═)—, [(C₁-C₆)alkyl]-NH—C(═O)—, [(C₁-C₆)alkyl]₂—N—C(═O)—, and non-, mono- and di-substituted amino wherein the substituents are independently selected from (C₁-C₆)alkyl, [(C₁-C₆)alkyl]-C(═O)—, [(C₁,-C₆)alkoxy]-C(═O)— and [(C₁-C₆)alkyl]-SO₂—;
(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or
R^{Y1} and R^{Y2} taken together with the carbon atom to which they are attached form spiropyrrolidinyl or spiropiperidinyl, both of which are optionally N-substituted with a substituent selected from (C₁-C₆)alkyl, (C₁-C₆)alkyl]-C(═O)—, [(C₁-C₆)alkyl]-C(═O)—(C₁-C₆)alkyl and aryl-(C═O)— wherein aryl is selected from phenyl and naphthyl; and R^{Y5} is defined as above;
R^{Y3} is hydrogen;
R^{Y4} is selected from
hydroxy;
(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]—C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
R^{Y6} and R^{Y7} are independently selected from
hydrogen;
(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;
hetrocyclyl-(CH₂)ₙ₆— wherein n6 is an integer selected from 0, 1, 2, 3 and 4 and said heterocyclyl is four to eight membered containing one to three hetero atoms independently selected from nitrogen, oxygen and sulfur, wherein said heterocyclyl is optionally substituted with one to three substituents independently selected from hydroxy; (C₁-C₆)alkyl; NH₂—C(O═)-; (C₁-C₆)alkyl-NH-C(═)—;(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and
hetroaryl-(CH₂)ₙ₇— wherein n7 is an integer selected from 0, 1, 2, 3 and 4 and said heteroaryl is five to ten membered containing one to three hetero atoms independently selected from nitrogen, oxygen and sulfur, wherein said heteroaryl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-,mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; or
R^{Y6} and R^{Y7} taken together with the nitrogen atom to which they are attached form a four to eight heterocyclyl optionally containing, in addition to the nitrogen atom, one to two additional hetero atoms independently selected from nitrogen, oxygen and sulfur, and said heterocyclyl is optionally substituted with one substituent selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—;
R^{Y81}, R^{Y82} and R^{Y83} are independently selected from R^{Y811} and R^{Y112}C(═O)— wherein R^{Y811} R^{Y812} are independently selected from
hydrogen;
hydroxy;
(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6},R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
said A is optionally substituted in the fused benzene rings with one to four substituents independently selected from
halo;
hydroxy;
mercapto;
phenyl;
(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independent selected from hydrogen, (C₁-C₆)alkyl, [(C₁-C₆)alkyl]-C(═O)—, [(C₁-C₆)alkoxy]-C(═O)— and C₆)alkyl]-SO₂—; and
Z is selected from
C(═O);
(CH₂)ₙ₈ wherein n8 is an integer selected from 0, 1 and 2; and
CHR^{Z1} wherein
R^{Z1} is selected from
carboxy;
(C₁-C₆)alkoxy-C(═O)—;
non-, mono- and di-substituted amino wherein the substituents are independently selected from (C₁-C₆)alkyl, [(C₁-C₆)alkyl]-C(═O)—, [(C₁-C₆)alkyl]-C(═O)—O— and [(C₁-C₆)alkyl]-SO₂—;
(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
[C(═O)—NR^{A11}R^{Z12}] wherein R^{Z11} and R^{Z12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—.

2. A compound according to claim 1 wherein
all R¹ are hydrogen
each R² is independently selected from hydrogen and halo;
X¹ is selected from (CH₂)ₙ₁ wherein n1 is an integer selected from 1, 2 and 3; O; NH; S; C(═O); SO₂; and N[(C₁-C₄)alkyl];
X² is selected from CH₂; O; NH; S; C(═O); SO₂; and N[(C₁-C₄)alkyl]; or
X¹ and X² taken together form CH═CH;
R^{Z1} is selected from
carboxy;

3. A compound according to claim 2 wherein
W¹ and W² are both CH₂;
A is AB wherein
both Y^{b} and Y^{c} are independently selected from
C(═O);
CR^{Y1} R^{Y2};
CR^{Y3}[C(═O)R^{Y4}];
CR^{Y3}[C(═O)NR^{Y6}R^{Y7}]; and
CR^{Y3}[NR^{Y6}R^{Y7}],

4. A compound according to claim 3 wherein
A is AB wherein
Y^{b} is CR^{Y3}[C(═O)NR^{Y6}R^{Y7}]; and
Y^{c} is selected from
CR^{Y1}R^{Y2};
CR^{Y3}[C(═))R^{Y4}];
CR^{Y3}[C(═O)NR^{Y6}R^{Y7}]; and
CR^{Y3}[NR^{Y6}R^{Y7}], wherein

5. A compound according to claim 4 wherein
Z is C(═O).

6. A compound according to claim 3 wherein
A is AB wherein
Y^{b} is CR^{Y1}R^{Y2}; and
Y^{c} is selected from
CR^{Y1}R^{Y2};
CR³[C(═O)R^{Y4}];
CR^{Y3}[C(═O)NR^{Y6}R^{Y7}]; and
CR^{Y3}[NR^{Y6}R^{Y7}]; or
Y^{b} and Y^{c} taken together form a group selected from CH₂-CH₂ and CH₂═CH₂;
Z is C(═O).

7. A compound according to claim 2 wherein
W¹ and W² are both CH₂;
A is AB wherein
Y^{b} is selected from
C(═O);
CR^{Y1}R^{Y2};
CR^{Y3}[C(═O)R^{Y4}];
CR^{Y3}[NR^{Y5}C(═O)R^{Y4}];
CR^{Y3}[C(═O)NR^{Y6}R^{Y7}]; and
CR^{Y3}[NR^{Y6}R^{Y7}];
Y^{c} is selected from
O;
S;
SO₂;
NH;
N[(C₁-C₆)alkyl] wherein said (C₁-C₆)alkyl is optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;
N—(CH₂)ₙ₃-heterocyclyl wherein n3 is an integer selected from 0, 1, 2 and 3, and said heterocyclyl contains from four to eight ring atoms one or two of which are independently selected from nitrogen, oxygen and sulfur;
N—(CH₂)ₙ₄-aryl wherein n4 is an integer selected from 0, 1, 2 and 3, and said aryl is selected from phenyl and naphthyl; and
N—(CH₂)ₙ₅-heteroaryl wherein n5 is an integer selected from 0, 1, 2 and 3, and said heteroaryl is a five to ten membered aromatic heterocyclyl containing from one to four hetero atoms independently selected from nitrogen, oxygen and sulfur;

8. A compound according to claim 1 wherein
all R¹ are hydrogen
each R² is independently selected from hydrogen and halo;
X¹ is selected from (CH₂)ₙ₁ wherein n1 is an integer selected from 1, 2 and 3; O; NH; S; C(═O); SO₂; and N[(C₁-C₄)alkyl];
X² is selected from CH₂; O; NH; S; C(═O); SO₂; and N[(C₁-C₄)alkyl]; or
X¹ and X² taken together form CH═CH;
A is AC

9. A compound according to claim 1 selected from 2,3-dihydro-1′-[3-(2-oxo-3,4-dihydro-1(2H)-quinolinyl)propyl]spiro[1H-indene-1,4′-piperidine] and 2,3-dihydro-1′-[3(3-methyl-2-oxo-3,4-dihydro-1(2H)-quinazolinyl)propyl]spiro[1H-indene-1,4′-piperidine]; or a salt thereof.
2,3-dihydro-1′-{3-[2-(N-methylaminocarbonyl)indolin-1-yl]-3-oxopropyl}spiro[1H-indene-1,4′-piperidine];
2,3-dihydro-1′-[3-(2-N,N-dimethylaminocarbonylindolin-1-yl)-3-oxopropyl]spiro[1H-indene-1,4′-piperidine];
2,3-dihydro-1′-[3-(2-morpholinocarbonylindolin-1-yl)-3-oxopropyl]spiro[1H-indene-1,4′-piperidine];
2,3-dihydro-1′-[3-(2-carbamoylindolin-1-yl)-3-oxopropyl]spiro[1H-indene-1,4′-piperidine] hydrochloride;
2,3-dihydro-1′-{3-[2-(1-ethylprrolydin-3-yl)aminocarbonylindolin-1-yl]-3-oxopropyl}spiro[1H-indene-1,4′-piperidine];
2,3-dihydro-1′-{3-[2-(S)-(N,N-dimethylaminoethyl)aminocarbonylindolin-1-yl]-3-oxopropyl}spiro[1H-indene-1,4′-piperidine];
2,3-dihydro-1′-{3-[2-(S)-(2-hydroxyethyl)aminocarbonylindolin-1-yl]-3-oxopropyl}spiro[1H-indene-1,4′-piperidine];
2,3-dihydro-1′-{3-[2-(S)-(2-aminoethyl)aminocarbonylindolin-1-yl]-3-oxopropyl}spiro[1H-indene-1,4′-piperidine];
2,3-dihydro-1′-{3-[2-(S)-(2-acetamidoethyl)aminocarbonylindolin-1-yl]-3-oxopropyl}spiro[1H-indene-1,4′-piperidine];
2,3-dihydro-1′-{3-[2-(S)-(2-methanesulfonamidoethyl)aminocarbonylindolin-1-yl]-3-oxopropyl}spiro[1H-indene-1,4′-piperidine];
2,3-dihydro-1 ′-[3-(2-(S)-N-methylaminocarbonylindolin-1-yl)-3-oxopropyl]spiro[1H-indene-1,4′-piperidine];
2,3-dihydro-1′-[3-(2-(S)-N,N-dimethylaminocarbonylindolin-1-yl)-3-oxopropyl]spiro[1H-indene-1,4′-piperidine];
2,3-dihydro-1 ′-{3-[2-(S)-(4-morpholinecarbonyl)indolin-1-yl]-3-oxopropyl}spiro[1H-indene-1,4′-piperidine]; and
2,3-dihydro-1′-[3-(2-(S)-aminocarbonylindolin-1-yl)-3-oxopropyl]spiro[1H-indene-1,4′-piperidine], or a salt thereof. 2,3-dihydro-1′-[3-(2-methoxycarbonylindolin-1-yl)-3-oxopropyl]spiro[1H-indene-1,4′-piperidine];
2,3-dihydro-1′-[3-(indolin-1-yl)-3-oxopropyl]spiro[1H-indene-1,4′-piperidine];
2,3-dihydro-1′-[3-(2-(S)-methoxycarbonylindolin-1-yl)-3-oxopropyl]spiro[1H-indene-1,4′-piperidine];
2,3-dihydro-1′-indolyl-3-oxopropyl}spiro[1H-indene-1,4′-piperidine];
2,3-dihydro-1′-[3-(2-hydroxymethylindolin-1-yl)-3-oxopropyl]spiro[1H-indene-1,4′-piperidine]; and
2,3-dihydro-1′-[3-(2-methoxymethylindolin-1-yl)-3-oxopropyl]spiro[1H-indene-1,4′-piperidine], or a salt thereof.
2,3-dihydro-1′-[3-(benzimidazol-2-one-1-yl)propyl]spiro[1H-indene-1,4′-piperidine];
2,3-dihydro-1′-[3-(benzothiazol-2-one-1-yl)propyl]spiro[1H-indene-1,4′-piperidine];
2,3-dihydro-1′-[3-(2-oxo-1,3-benzoxazol-3(2H)-yl)propyl]spiro[1H-indene-1,4′-piperidine];
2,3-dihydro-1′-[3-(2-hydroxymethylbenzimidazol-1-yl)-3-oxopropyl]spiro[1H-indene-1,4′-piperidine];
2,3-dihydro-1′-[3-(3-ethylbenzimidazol-2-one-1-yl)propyl]spiro[1H-indene-1,4′-piperidine];
2,3-dihydro-1′-[3-(2-acetamidobenzimidazol-1-yl)propyl]spiro[1H-indene-1,4′-piperidine];
2,3-dihydro-1′-{3-[3-(2-hydroxyethyl)benzimidazol-2-one-1-yl)propyl}spiro[1H-indene-1,4′-piperidine];
2,3-dihydro-1′-{3-[3-(2-aminoethyl)benzimidazol-2-one-1-yl)propyl}spiro[1H-indene-1,4′-piperidine]; and
2,3-dihydro-1′-{3-[3-(2-acetamidoethyl)benzimidazol-2-one-1-yl)propyl}spiro[1H-indene-1,4′-piperidine], or a salt thereof.

10. A pharmaceutical composition comprising an effective amount of a compound of claim 1 and a pharmaceutically acceptable carrier for treating a disease or medical condition mediated by ORL1-receprot and its endogeneous ligand in a mammal including a human.

11. A pharmaceutical composition according to claim 10 wherein the compound has one of selectivity for ORL-1 receptor, antagonist effect for ORL-1 receptor, or is selective iantagonist for ORL-1 receptor.

12. A pharmaceutical composition according to claim 10, wherein the disease or medical condition is selected from pain; eating disorders including anorexia and bulimia; anxiety and stress conditions; immune system diseases; locomotor disorder; eating disorder; memory loss, cognitive disorders and dementia including senile dementia and those diseases caused by Alzheimer's disease, Perkinson's disease or other neurodegenerative pathologies; epilepsy or convulsion and symptoms associated therewith; a central nervous system disorder related to gulutamate release action, anti-epileotic action, disruption of spatial memory, serotonin release, anxiolytic action, mesolimbic dopaminergic transmission, rewarding propaerties of drug of abuse, modulation of striatal and glutamate effects on locomotor activity; cardiovascular disorders hypotension, bradycardia and stroke; renal disorders including water excretion, sodium ion excretion and syndrome of inappropriate secretion of antidiuretic hormone (SlADH); gastrointestinal disorders; airway disorders including adult respiratory distress syndrome (ARDS); autonomic disorders including suppression of micturition reflex; metabolic disorders including obesity; cirrhosis with ascites; sexual dysfunctions; and altered pulmonary function including obstructive pulmonary disease.

13. A method for treating or preventing a disease or condition in a mammal including a human, which disease or condition is mediated by ORL-1 receptor and its endogeneous ligand, comprising administering an effective amount of a compound of claim 1 to a mammal including a human, which suffered from such disease or condition wherein said compound has one of selectivity for ORL-1 receptor, antagonist effect for ORL-1 receptor, or is a selective antagonist for ORL-1 receptor.

14. A method for treating or preventing a disease or medical condition according to claim 11 wherein said disease or condition is selected from pain; eating disorders including anorexia and bulimia; anxiety and stress conditions; immune system diseases; locomotor disorder; eating disorder; memory loss, cognitive disorders and dementia including senile dementia and those diseases caused by Alzheimer's disease, Perkinson's disease or other neurodegenerative pathologies; epilepsy or convulsion and symptoms associated therewith; a central nervous system disorder related to gulutamate release action, anti-epileotic action, disruption of spatial memory, serotonin release, anxiolytic action, mesolimbic dopaminergic transmission, rewarding propaerties of drug of abuse, modulation of striatal and glutamate effects on locomotor activity; cardiovascular disorders hypotension, bradycardia and stroke; renal disorders including water excretion, sodium ion excretion and syndrome of inappropriate secretion of antidiuretic hormone (SlADH); gastrointestinal disorders; airway disorders including adult respiratory distress syndrome (ARDS); autonomic disorders including suppression of micturition reflex; metabolic disorders including obesity; cirrhosis with ascites; sexsual dysfunctions; and altered pulmonary function including obstructive pulmonary disease.
